(19) United States
(12) Patent Application Publication
(10) Pub. No.: US 2023/0390031 A1
(43) Pub. Date: Dec. 7, 2023

(54) SYSTEMS AND METHODS FOR LIBRARY-BASED TOOTH SELECTION IN DIGITAL DENTAL APPLIANCE DESIGN

(71) Applicant: VOYAGER DENTAL, INC., EDEN PRAIRIE, MN (US)

(72) Inventors: Michael C. Marshall, Edina, MN (US); John Madden, Minneapolis, MN (US); Stephen B. Siegfried Floe, Chanhassen, MN (US)

(21) Appl. No.: 18/328,461

(22) Filed: Jun. 2, 2023

Related U.S. Application Data
(60) Provisional application No. 63/348,217, filed on Jun. 2, 2022.

(51) Int. Cl. A61C 9/00; G06T 19/20

(52) U.S. Cl. CPC A61C 9/0053; G06T 19/20; A61C 13/34

(57) ABSTRACT

Disclosed are systems and methods for selecting a tooth library for auto-designing a dental appliance. A method includes receiving, by a computer system, patient tooth data including a scan of the patient's teeth, identifying markers on the teeth in the patient tooth data, such as opposing tooth edges, determining teeth measurements based on the markers, such as determining a width between the opposing tooth edges, and selecting, from a static tooth library data store, a subset of tooth libraries based on the teeth measurements and based on: identifying at least one tooth library having respective tooth width measurements within a threshold range of the determined width of the tooth in the patient tooth data, and adding the identified at least one tooth library to the subset of tooth libraries. The method also includes selecting a candidate tooth library from the subset based on the candidate tooth library satisfying dental-appliance-fit criteria.

### INCORPORATION BY REFERENCE

This application claims priority to U.S. Provisional Application Ser. No. 63/348,217 entitled “Auto-Denture Design System for Denture Replacement,” filed on Jun. 2, 2022, the disclosure of which is incorporated by reference in its entirety.

### TECHNICAL FIELD

This document generally describes technology and techniques related to an auto-design system with library-based tooth selection techniques for selecting tooth libraries in designing dental appliances for a dental patient, the dental appliances including but not limited to dentures, veneers, and dental implants.

### BACKGROUND

A tooth library can include varieties of predefined, standard teeth, including but not limited to individual teeth and sets or groups of teeth that are sized to work well with each other and together. Unlike scalable tooth models, a tooth library provides a balanced selection of teeth that are sized relative to each other and look good together. Teeth libraries can be generated for different types of teeth. Teeth libraries can be fully anatomical. The teeth libraries can be reproduced and fitted to different patients' mouths based on the patients' needs and a type of dental appliance being designed and/or manufactured for the patients. The dental appliance can include but is not limited to dentures, veneers, dental implants, and/or dental inserts. The teeth libraries can be loaded into teeth modeling software and manipulated by a user to determine an appropriate fit and arrangement of the teeth libraries for a particular patient. The user can select the teeth libraries to use based on manually reviewing stored teeth libraries and visually identifying which of the teeth libraries appear to be a best fit for the particular patient's condition.

A denture is a dental prosthesis that is made to replace missing teeth. Dentures are often supported by the surrounding soft and hard tissue of the oral cavity. For example, a denture may be designed to fit over and be supported by a patient's gum tissue. Dentures may include a denture base region that is formed from an acrylic material and colored to appear similar to gum tissue. Denture teeth formed from acrylic or other materials may be secured to the denture base.

A veneer is a tooth-colored shell that can be attached to a front surface of a tooth to improve appearance of the tooth and an overall appearance of a patient's teeth. Veneers can be used to correct various dental issues, such as stained, chipped, crooked, and/or damaged teeth. Veneers can also help conceal uneven spaces between teeth. Veneers can be porcelain or composite resin. Porcelain veneers can, for example, be a thin shell that is custom-made to fit on the patient's tooth, strong and long-lasting, natural-looking, requiring less enamel removal than a crown or cap, and/or won't stain as easily. Composite resin veneers can, for example, be made from a tooth-colored filling material bonded to the patient's tooth, be repaired easily and quickly if damaged, fast to install in the patient's mouth, require less enamel removal than crowns, and/or be an affordable option. Veneers can be prepared and shaped for the patient's mouth, then bonded and sculpted using a color suitable for the patient.

A dental implant, dental insert, or other type of dental appliance can include crowns, caps, bridges, other types of dentures, etc. The dental implant can be a prosthesis that interfaces with a bone of the patient's jaw and/or skull to support the prosthesis. The dental implant can be surgically implanted into the patient's jaw to restore their ability to chew and/or to restore their overall teeth appearance. The dental implant can be designed and fitted for the patient's mouth.

### SUMMARY

This document generally describes systems, methods, and computer-based techniques for selecting tooth libraries to be used in designing dental appliances for a patient. The dental appliances can include, but are not limited to, dentures, veneers, dental implants, inserts, crowns, and/or caps. The disclosed technology can implement computer-automated techniques, such as identifying and annotating tooth landmarks in patient tooth data (e.g., 3D scans of the patient's mouth, 2D image data of the patient's mouth), determining tooth measurements based on the identified and annotated landmarks, selecting tooth libraries from a static tooth library data store that have similar measurements as the determined measurements for the particular patient, scoring the selected tooth libraries using a fitting algorithm (e.g., best fit to the measurements for the particular patient), and selecting a candidate tooth library (or candidate tooth libraries) having a respective score that satisfies one or more selection criteria. The measurements can be determined using one or more machine learning models and/or artificial intelligence (AI) algorithms and techniques, as described herein. The measurements can include measurements of teeth widths, individual tooth widths, aggregated teeth widths, lengths of incisors, and other types of measurements that may be specific to the type of dental appliance being designed for the particular patient. The selected tooth libraries can be scored based on a similarity of their respective measurements to the determined measurements for the particular patient. For example, tooth libraries that have respective teeth widths within a threshold range of the teeth widths for the particular patient can be selected and scored higher than tooth libraries that have respective teeth widths that do not fall within the threshold range. The selected and scored tooth libraries can be narrowed to one or more of the candidate tooth libraries based on measuring and comparing central incisor lengths of the tooth libraries to the measurements for the patient and comparing shape of the tooth libraries to the measurements for the patient. Various other factors and/or criteria can be used to select the candidate tooth library or libraries.

The candidate tooth library can then be used in a variety of contexts, such as automatically designing dental appliances for patients, generating imagery (e.g., images, animation, videos, 2D and/or 3D models), showing the application of the candidate tooth libraries for dental appliances and/or for the specific patient, and/or other appropriate uses. For example, a candidate tooth library can also be used by a relevant user, such as a dentist, oral surgeon, orthodontist, or other technician, to manually design the dental appliances for the particular patient. For instance, the candidate tooth library (and/or other tooth libraries in the data store) can be outputted in one or more graphical user interfaces (GUIs) at a user device of the relevant user. The relevant user can then select and manipulate the presented candidate tooth library as well as the auto-design of the dental appliances for the particular patient.

Models of dental appliances can be used to create dental appliances specific to each patient. The dental appliances may need to be properly fitted to the soft tissue in a patient's mouth for comfort and proper function or more generally to contours of the patient's mouth and/or jaw. A physical appearance, including shape, color, and pattern of the dental appliances can also be important to the patient. Designing dental appliances from a scan of the patient's mouth can be time consuming for a dentist or technician. Each dental appliance may require a custom design to ensure patient fit and satisfaction. Conventional methods for producing these custom designs can be time-consuming and imprecise, and the results can be technician-dependent as much of the design process relies on a technician making selections and design choices based on visual inspection of tooth libraries and/or existing scans or models of the dental appliances. The resulting dental appliances may be an imperfect fit, which impacts the patient experience. Rework and readjustments to correct the fit can drive cost and inefficiency for patients, dentists, dental offices, laboratories, and other relevant users.

One or more embodiments described herein can include a method for selecting a candidate tooth library for auto-designing a dental appliance for a patient, the method including: receiving, by a computer system, patient tooth data, the patient tooth data including a scan of a patient's teeth, identifying, by the computer system, one or more markers on the teeth in the patient tooth data, the markers including at least opposing edges on a tooth, determining, by the computer system, teeth measurements based on the identified markers, where determining the teeth measurements can include determining a width between the opposing edges on the tooth, and selecting, by the computer system and from a static tooth library data store, a subset of tooth libraries based on the teeth measurements. Selecting the subset of tooth libraries based on the teeth measurements can include: identifying at least one tooth library having respective tooth width measurements within a threshold range of the determined width of the tooth in the patient tooth data, and adding the identified at least one tooth library to the subset of tooth libraries. The method can also include selecting, by the computer system, a candidate tooth library from the subset of the tooth libraries based on the candidate tooth library satisfying one or more dental-appliance-fit criteria and returning, by the computer system, the candidate tooth library for the patient.

The embodiments described herein can optionally include one or more of the following features. For example, identifying, by the computer system, one or more markers on the teeth in the patient tooth data can include: applying at least one model to the patient tooth data, the model having been trained with machine learning techniques to identify and label, as the markers, at least one of opposing tooth edges, a cusp, a cusp tip, and a marginal ridge. Determining, by the computer system, teeth measurements based on the identified markers can include determining lengths of incisors for at least one of the teeth. Determining, by the computer system, teeth measurements based on the identified markers can include determining a shape of at least one of the teeth. Determining, by the computer system, teeth measurements based on the identified markers can include determining at least one interproximal (IP) contact between the teeth. Selecting, by the computer system and from a static tooth library data store, a subset of tooth libraries can also be based on the teeth measurements. Selecting the subset of tooth libraries based on the teeth measurements can include: aggregating tooth width measurements for the teeth in the patient tooth data, identifying at least one tooth library having a respective aggregate tooth width measurement within a threshold range of the aggregated tooth width measurements for the teeth in the patient tooth data, and adding the identified at least one tooth library to the subset of tooth libraries.

In some implementations, the method can include scoring, by the computer system, each tooth library in the subset of tooth libraries. The computer system can assign a score above a threshold value to the tooth library when the respective tooth width measurements are within a threshold scoring range of the determined width of the tooth in the patient tooth data. Scoring can include assigning a score below the threshold value to the tooth library when the respective tooth width measurements exceed the threshold scoring range of the determined width of the tooth in the patient tooth data. The threshold scoring range can be based on a type of the dental appliance being designed for the patient, the type of the dental appliance being at least one of dentures, veneers, dental implants, and dental inserts. A threshold scoring range for the dentures can be greater than threshold scoring ranges for the veneers, the dental implants, and the dental inserts. Selecting, by the computer system, a candidate tooth library from the subset of the tooth libraries can also be based on a score of the candidate tooth library satisfying the one or more dental-appliance-fit criteria.

Sometimes, selecting, by the computer system, a candidate tooth library from the subset of the tooth libraries can include selecting the candidate tooth library having respective incisor lengths within a threshold length range of determined incisor lengths of the teeth in the patient tooth data. Selecting, by the computer system, a candidate tooth library from the subset of the tooth libraries can include selecting the candidate tooth library having respective tooth shapes within a threshold shape range of determined shapes of the teeth in the patient tooth data. Selecting, by the computer system, a candidate tooth library from the subset of the tooth libraries can include selecting the candidate tooth library having a highest respective score amongst the subset of tooth libraries. Sometimes, selecting, by the computer system, a candidate tooth library from the subset of the tooth libraries can include selecting the candidate tooth library having a respective score that exceeds a threshold score value.

As another example, selecting, by the computer system, a candidate tooth library from the subset of the tooth libraries can include: removing, from the subset of the tooth libraries, at least one tooth library having respective incisor lengths that exceed a threshold length range of determined incisor lengths of the teeth in the patient tooth data, determining whether the subset of the tooth libraries comprises more than one tooth library, and removing, from the subset of the tooth libraries and based on determining that the subset includes more than one tooth library, at least another tooth library having respective tooth shapes within a threshold shape range of determined shapes of the teeth in the patient tooth data.

Returning the candidate tooth library can cause the computer system to auto-design a dental appliance for the patient based at least in part on the candidate tooth library, the dental appliance being at least one of dentures, veneers, a dental implant, and a dental insert. Returning, by the computer system, the candidate tooth library can include transmitting the candidate tooth library to a user device of a relevant user for presentation in a graphical user interface (GUI) at the user device. The method can also include receiving, by the computer system from the user device, user input indicating selection of another tooth library from the subset of tooth libraries, and auto-designing, by the computer system, a dental appliance for the patient based on the user-selected tooth library. Sometimes, presenting the candidate tooth library in the GUI can include displaying the teeth of the candidate tooth library in a digital dental appliance model using a color map. The color map can illustrate differences between the teeth of the candidate tooth library and teeth of the digital dental appliance model.

The disclosed techniques may provide one or more advantages. For example, the disclosed technology can use less computing power and resources to automatically label and measure patient tooth data, assess many different preexisting tooth libraries, and identify and select at least one preexisting tooth library that may work best for the particular patient's dental appliance design. More particularly, comparing tooth libraries based on teeth width measurements can provide for more computationally efficient (e.g., use fewer CPU cycles, require less RAM for identifying appropriate library) and accurate selection of tooth libraries for the particular patient, which can additionally be done without requiring a user's visual inspection, manual review, and manual manipulation of hundreds if not thousands of tooth libraries from which to select. For example, the disclosed technology can be used to identify an appropriate tooth library for a particular patient and their unique dimensions (e.g., dimensions of their teeth, gums, mouth, face, etc.) so that it fits the patient well and provides a pleasing visual aesthetic for the patient, while also performing these identifications in a manner that is computationally efficient yet still takes into consideration the patient's 3D geometry. Instead of using image analysis techniques that may identify tooth libraries that provide a good visual aesthetic for a patient but which may not account for the patient's full 3D geometry, the disclosed technology can provide both beneficial outcomes and in a manner that can avoid some of the more computationally resource intensive operations involved with image-based analysis. Additionally, the disclosed technology can provide better and more accurate tooth library selection over human-based selection, and in a manner that is more efficient and cost effective.

As another example, using software to automate portions of the dental appliance design process can improve efficiency over technician development of dental appliance designs based on visual identification of dental appliance landmarks. A CT scanning device, CBCT scanning device, or other scanning device can quickly produce a scan of a patient's teeth, which can be used by a computer program or computer system more generally to automatically identify a matching tooth library, determine various landmarks in a digital model produced from the scan, and position and align teeth from the tooth library in the digital model. The process for designing of the dental appliances would be significantly longer if a technician was required to complete these steps based on visual inspection and manual manipulation of the tooth libraries and digital models. The disclosed technology can curtail and shorten the time between scan, design, and installation.

Automation of portions of the dental appliance design process can enable the standardization of a process of producing a digital dental appliance design. Landmarks and best-fits can be efficiently determined using the disclosed technology and iterative best fit algorithms with greater accuracy than a technician can produce. The automatically produced designs can be of higher-quality than technician-produced designs because they may very closely replicate a design of existing dental appliances. This can reduce variability in designs produced by different technicians who produce designs by visual inspection.

Moreover, automating the design process can significantly speed up the process by identifying, selecting, and suggesting tooth libraries that are a close match to the patient's teeth and/or desired dental appliances, and suggesting appropriate positions and orientations of the teeth in the design. The automated techniques provide for less work to be performed by a technician, and the technician can simply review the suggestions and make any minor adjustments to the digital model rather than create the entire model manually and/or from scratch. This can decrease the amount of technician time required for creating a digital dental appliance design and increase throughput of designs. Designs for dental appliances can be sent for manufacturing more quickly, and patients can receive their dental appliances quickly and at less cost due to the automated process reducing the time required for designing. Similarly, automating the techniques described herein allows for less-skilled technicians or other users to review and design dental appliances.

Additional advantages will be apparent to the person of skill in the art based on the figures, description, and claims herein.

### DESCRIPTION OF THE DRAWINGS

FIG. 1A is a conceptual diagram of a system for selecting tooth libraries for designing dental appliances for a patient.

FIG. 1B illustrates an example digital dental model with a selected tooth library for a tooth implant.

FIG. 1C is a conceptual diagram of system components for selecting tooth libraries.

FIGS. 2A, 2B, and 2C are a flowchart of a process for selecting tooth libraries for designing a denture for a patient.

FIGS. 3A and 3B are a flowchart of a process for selecting tooth libraries for designing veneers for a patient.

FIGS. 4A and 4B are a flowchart of a process for selecting tooth libraries for designing dental implants for a patient.

FIG. 5 is a flowchart of a process for training at least one model to be used in selecting tooth libraries for designing dental appliances for patients.

FIG. 6 is a flowchart of an example method for automatic design of a replacement denture.

FIGS. 7A and 7B illustrate example user interface screens that may be generated during a denture design process of positioning selected tooth library teeth in a digital denture model and automatically aligning and leveling each of the selected tooth library teeth.

FIGS. 8A, 8B, and 8C illustrate example user interface screens that may be generated during a denture design process of automatically aligning and leveling each selected tooth library teeth.

FIG. 9A illustrates example libraries of digital denture teeth.

FIG. 9B illustrates differences between the digital denture teeth of FIG. 9A with an overlay color map.

FIG. 10 is a system diagram of one or more components that can be used to perform the techniques described herein.

FIG. 11 is an example architecture of a computing device, which can be used to implement aspects according to the present disclosure.

Like reference symbols in various drawings indicate like elements.

### DETAILED DESCRIPTION

This document generally describes techniques for automatic and accurate selection of a candidate tooth library for use in designing dental appliances for a particular patient. The disclosed technology can provide automatic detection of landmarks of teeth in patient tooth data, which can enable efficient and accurate selection of tooth libraries having a close match to size and/or shape of the patient's teeth. Using the disclosed technology, the selected tooth libraries can be automatically placed and adjusted in a digital dental appliance model for the patient. The model can be used to accurately and efficiently design the dental patient's dental appliance for fabrication and manufacturing. The computer-automated techniques described herein can be more efficient than selection by a technician or other relevant user based on visual inspection of many tooth libraries. The computer-automated techniques also can reduce variability in dental appliance designs that may arise when different technicians visually inspect and manually manipulate tooth libraries and/or dental appliance design models. The dental appliance design that results using the disclosed technology can be used to fabricate dental appliances that provide improved fit, function, and aesthetics for the particular patient.

Different technicians may make design selections differently or based on different criteria, resulting in variation in dental appliances designed by the different technicians. For example, it can be difficult for the technician to visually distinguish dimensional differences between teeth of varying shapes, and requiring the technician to make these determinations can create challenges. For example, the technician's guesswork in determining and selecting tooth libraries that match the patient's tooth data can cause differences in fit and function for the particular patient. The technician selection of tooth libraries can introduce variability in an output design and manufactured dental appliances. The dental appliances may also look different if the selected tooth libraries vary from an expected or existing dental appliance design for the patient, and can fail to meet expectations of the patient.

By using software and other computer-automated techniques employing iterative fitting algorithms, machine learning models, and/or AI algorithms to aid in the design of dental appliances, the process of designing dental appliances can be significantly improved. The speed with the disclosed technology can identify one or more matching tooth libraries can be faster and more accurate than manual visual identification by technicians. The disclosed technology can take multiple landmarks of the digital model into account in determining the appropriate tooth library to select, making the dental appliances more likely to achieve fit, function and appearance criteria for the particular patient.

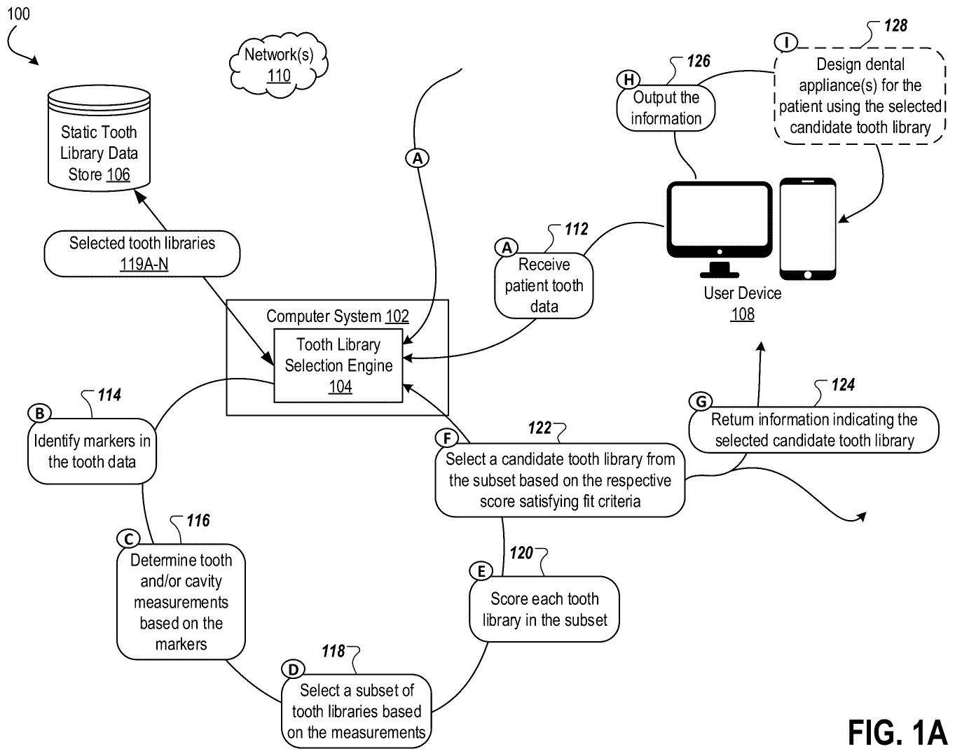

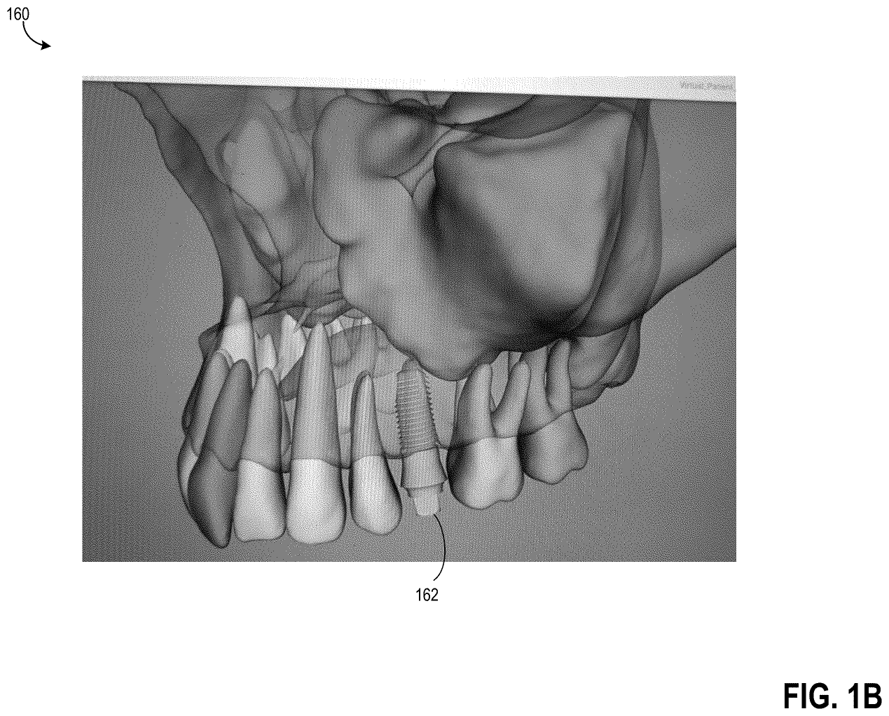

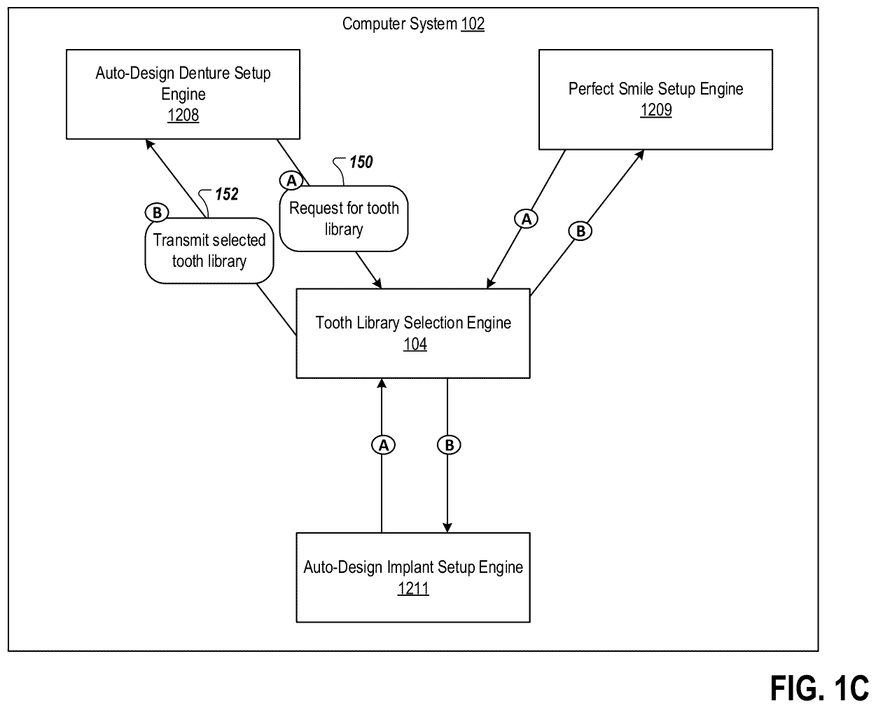

Referring to the figures, FIG. 1A is a conceptual diagram of a system 100 for selecting tooth libraries for designing dental appliances for a patient. Machine learning models, AI algorithms, and other computer-automated techniques can be used to measure a particular patient's teeth and use those measurements to select one or more candidate tooth libraries for the patient. For example, a 2D image can be taken of the patient's mouth, dentition, dentures, smile, etc. Using AI algorithms and/or machine learning models, the system 100 can process the 2D image to recognize and label markers, lines, and geometry of the patient's teeth, mouth, jaw, and face. The recognized features can then be used by the system 100 to select appropriate tooth libraries to auto-design dental appliances for the patient.

As an illustrative example, a model can be trained with machine learning techniques to identify and label different markers and/or geometry of patients' teeth, mouth, jaw, and facial features. The model can be trained to determine measurements amongst the different identified and labeled markers for a patient. The model can return the determined measurements, which can be used by a computer system to identify and select a tooth library that has most similar or same measurements (and/or measurements within a threshold range of the determined measurements for the patient, such as within 1 mm).

As another example, the model can be trained to identify different tooth shapes in patient image data, then output the identified tooth shapes and/or confidence values/scores for those tooth shapes. The computer system can then identify and select a tooth library that has most similar or same tooth shapes and/or confidence values/scores. In some implementations, the models described herein can be trained to output a selected tooth library based on the determined measurements and/or shapes for the patient.

Conventionally, scalable tooth libraries may be used. However, scalable tooth libraries can cause variations in size, fit, and appearance of any teeth applied to a design of dental appliances for the patient. The disclosed technology, therefore, provides improved techniques for selecting candidate tooth libraries that would allow for accurate design of dental appliances for the patient.

The system 100 can include a computer system 102 having at least a tooth library selection engine 104, a user device 108, and a static tooth library data store 106. The components of the system 100 can communicate (e.g., wired and/or wirelessly) via network(s) 110.

In brief, the computer system 102 can be any type of computing system, cloud-based computing system, computing device, and/or network of computer systems. The computer system 102 can be remote from dental offices or other locations where a patient may go to get fitted for and receive dental appliances. In some implementations, the computer system 102 can be in the dental office and/or part of the user device 108 or other computer system in the dental office. The computer system 102 can be configured to generate dental appliance designs unique to each patient based on processing patient-specific data, selecting tooth libraries that satisfy one or more selection criteria, and using the selected tooth libraries to design dental appliances for the patient. The tooth library selection engine 104 can be configured to select a candidate tooth library for the particular patient and based on the patient tooth data, as described further herein. For further discussion about designing the dental appliances, refer to U.S. Provisional Application Ser. No. 63/348,217 entitled “Auto-Denture Design System for Denture Replacement” (the '217 application), filed on Jun. 2, 2022, the disclosure of which is incorporated by reference in its entirety.

The static tooth library data store 106 can be any type of database, data store, data repository, and/or cloud-based storage system. The data store 106 can store many different tooth libraries that have been predefined and/or previously generated. The tooth libraries can be generic and applicable to all patients. In some implementations, the tooth libraries can be generated for particular types of teeth, particular groups of teeth, particular purposes (e.g., dental implants, dental inserts, caps, crowns, veneers, dentures), particular patient demographics (e.g., age, gender, dental condition), etc. The tooth libraries can each contain data or metadata, which further can be used with the disclosed techniques. For example, each tooth library can include a predefined coordinate system. The coordinate system can then be used by the computer system 102 in determining one or more measurements of the respective tooth and/or arranging and setting up the tooth relative to other teeth for the patient's dental appliance design. Each tooth library can include additional or other information, including but not limited to tooth measurements, color data, shape data, texture data, etc.

The user device 108 can be any type of computing device including but not limited to smartphones, tablets, laptops, computers, mobile phones, mobile devices, and/or wearable devices. The user device 108 can be used by a relevant user, such as a technician, dentist, and/or orthodontist. The user device 108 can be configured to present and output information in GUIs about selected tooth libraries and/or auto design of the dental appliances to the relevant user. The user device 108 can also be configured to receive user input indicating selection of tooth libraries and/or manual manipulation of the design of the dental appliances. Refer to description throughout this disclosure and at least the '217 application for additional information about the GUIs presented at the user device 108.

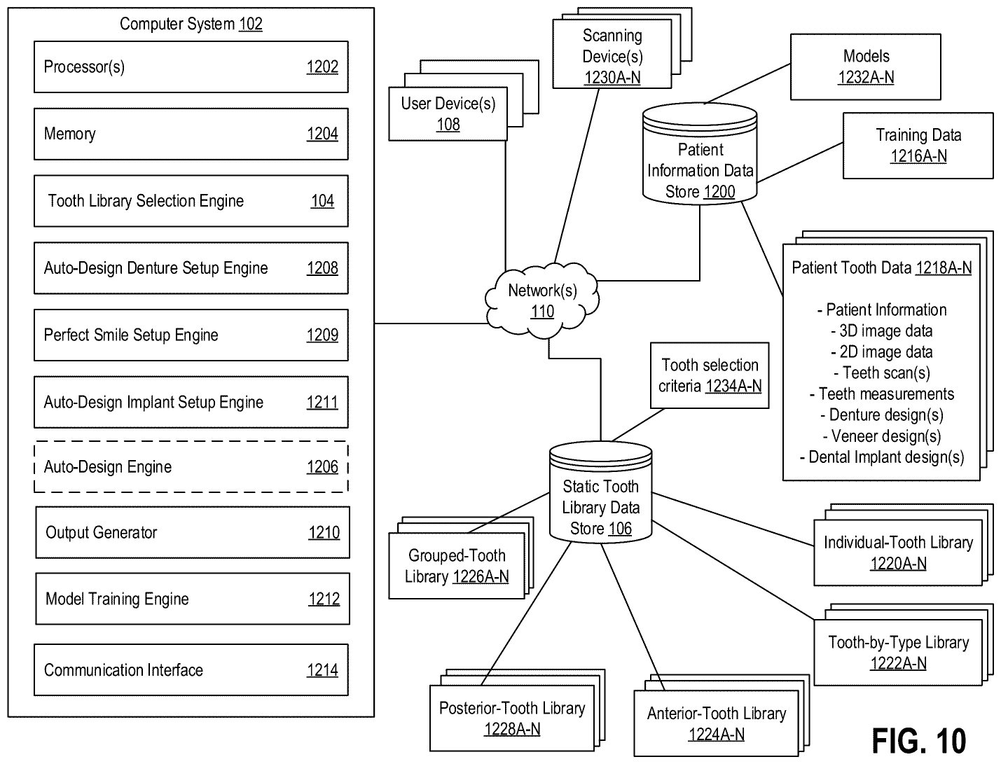

Refer to FIG. 10 for further discussion about the components of the system 100.

Still referring to FIG. 1A, the tooth library selection engine 104 can receive patient tooth data in block A (112). This data can be received from the user device 108 or any other computing system or device. For example, the patient's mouth and/or teeth can be scanned using a scanning device or 3D imaging device in a dental office. The scanning device or 3D imaging device can transmit, via the network(s) 110, the scan data to the engine 104. As another example, the scan data can be transmitted first to the user device 108 or a patient data store and then transmitted to the engine 104. The engine 104 can request the scan data or other patient tooth data once the user provides user input at the user device 108 indicating a desire to design a dental appliance for a particular patient. Other times, the engine 104 can automatically receive the patient tooth data when the patient tooth data is generated (e.g., when scans or images are taken of the patient's teeth and/or mouth) and/or at predetermined times (e.g., every 15 minutes, every 30 minutes, every 1 hour, every 5 hours, every 12 hours, every 24 hours). In some implementations, the engine 104 can retrieve the patient tooth data from a data store.

The engine 104 can identify one or more markers in the tooth data in block B (114). The markers can include information about each individual tooth in the data and/or sets or groups of teeth (e.g., canines, incisors, molars, upper teeth, lower teeth, posterior teeth, anterior teeth). The information can be landmarks, including but not limited to edges of one or more teeth, center points of the teeth, cusp tips of the teeth, etc. To identify the markers, the engine 104 can apply one or more machine learning models to the patient tooth data. The models can be trained to identify particular types of landmarks or markers and label/annotate them as such. The engine 104 can apply a model that was trained to identify a particular type of markers needed for selecting tooth libraries for a particular type of dental appliance. For example, the engine 104 can apply a model for identifying tooth edges, which can be used for selecting tooth libraries for designing dentures, then apply another model for identifying tooth lengths, which can be used for selecting tooth libraries for designing veneers. Various other techniques described herein can be used for identifying the markers in the patient tooth data.

Based on the identified markers, the engine 104 can determine tooth and/or cavity measurements in the patient tooth data (block C, 116). For example, 2 opposing edges can be identified for a particular tooth in block B (114). The engine 104 can then measure a distance between the 2 opposing edges to determine a width of the tooth. In some implementations, the engine 104 can determine an aggregate width across a set of teeth, such as an entire upper arch, posterior teeth, anterior teeth, canines, incisors, etc. In some implementations, the engine 104 can determine a length measurement of each tooth or particular teeth. The engine 104 can determine other measurements of the teeth (e.g., for designing dentures, veneers, dental implants, and/or dental inserts) and/or teeth cavities (e.g., for designing dental implants and/or dental inserts). In some implementations, one or more of the models described in reference to block B (114) can also be trained to determine the tooth and/or cavity measurements and provide those measurements as output.

The engine 104 can select a subset of tooth libraries from the static tooth library data store 106 based on the measurements (block D, 118). The engine 104 can retrieve tooth libraries from the data store 106 having measurements that are within threshold ranges or values of the determined measurements for the patient tooth data. The engine 104 can also use one or more selection criteria to select tooth libraries in block D (118). For example, the engine 104 can select tooth libraries that have been generated for denture design if dentures are being designed for the particular patient. Accordingly, selected tooth libraries 119A-N can be transmitted to the engine 104 from the data store 106.

In block E (120), the tooth library selection engine 104 can score each tooth library in the subset. The engine 104 can score the tooth libraries using a fitting algorithm. The fitting algorithm can include a best fit technique and iterative closest point (ICP) algorithm. For example, using existing denture teeth for the patient, the computer system can perform a best fit algorithm of teeth in the tooth libraries. Once the computer system identifies tooth libraries having the best or closest fit to the existing denture teeth for the patient, the computer system can use the ICP algorithm to identify how close of a fit (e.g., average distance, standard deviation, mean of all points for the teeth) the identified tooth libraries are with the existing denture teeth for the patient.

As another example, the engine 104 can score the tooth libraries based on respective measurements, such as tooth widths, shapes, and/or heights. The engine 104 can compare the measurements of the tooth library in the subset to the measurements of the patient tooth data. The computer system can aggregate tooth widths for the tooth library and aggregate tooth widths for the patient tooth data, then compare the aggregated tooth widths to each other. In some implementations, the computer system can compare tooth widths on an individual tooth basis (rather than aggregating tooth width values for sets of teeth). The closer the measurements of the tooth library to the measurements of the patient tooth data, the higher score value can be assigned to the tooth library (thereby indicating that the tooth library is a good or better fit for the particular patient). The greater a difference between the measurements of the tooth library and the measurements of the patient tooth data, the lower score value can be assigned to the tooth library (thereby indicating that the tooth library is less of a good or best fit). Score values can be assigned on any variety of integer or numeric scales, such as 0 to 5, 1 to 10, 1 to 100, 0 to 100, etc. Score values can also be assigned as Boolean and/or string values.

The engine 104 can select a candidate tooth library from the subset based on the respective score satisfying one or more fit criteria (block F, 122). The engine 104 can select the candidate tooth library having the highest score value in the subset. The engine 104 can select at least one of the tooth libraries in the subset having a score value that exceeds some threshold score value.

In some implementations, the engine 104 can select the candidate tooth library using one or more additional or other selection criteria. For example, the engine 104 can narrow down which tooth libraries to keep in the subset based on measuring and comparing incisor lengths of the teeth in the tooth libraries to the determined incisor lengths of the patient tooth data. The engine 104 can keep tooth libraries in the subset that (i) have respective tooth widths within a threshold range of the measured tooth widths of the patient tooth data and (ii) have respective incisor lengths that are within another threshold range of the measured incisor lengths of the patient tooth data. Tooth libraries that do not satisfy (i) and (ii) can be removed from the subset.

Additionally or alternatively, the engine 104 can narrow down which tooth libraries to keep in the subset based on comparing tooth shapes. Generally, patients can have square, tapered, and/or ovoid shaped teeth. A relevant user, such as a technician or dentist, can provide user input to the engine 104 indicating a tooth shape for the particular patient. In some implementations, the patient tooth data can already be annotated with the tooth shape for the particular patient. The engine 104 can then narrow down which tooth libraries remain in the subset, or which is selected as the candidate tooth library, based on the abovementioned tooth shape information. In yet some implementations, at least one model can be trained with machine learning techniques to recognize the tooth shape from existing dentures or natural existing anatomy of patients in patient tooth data, patient scan data, and/or patient image data. To automatically identify the tooth shape, for example, the model can be trained to generate a bounding box around each tooth and recognize geometric shapes, datums, markers, or other identifiers that can be used to classify shape of each tooth.

The engine 104 can keep tooth libraries in the subset that (i) have respective tooth widths within a threshold range of the measured tooth widths of the patient tooth data and (ii) have respective tooth shapes that are similar to or within expected tooth shape ranges as the determined tooth shapes of the patient tooth data. The engine 104 can then remove any tooth libraries from the subset that do not satisfy the width and shape comparisons. As yet another example, the engine 104 can keep tooth libraries in the subset that (i) have respective tooth widths within a threshold range of the measured tooth widths of the patient tooth data, (ii) have respective incisor lengths that are within another threshold range of the measured incisor lengths of the patient tooth data, and (iii) have respective tooth shapes that are similar to or within expected tooth shape ranges as the determined tooth shapes of the patient tooth data. Any tooth library that does not satisfy the width, length, and shape comparisons can be removed from the subset. As a result, the engine 104 can narrow down the subset to at least one candidate tooth library, which can be considered a best fit for the particular patient.

The engine 104 can return information indicating the selected candidate tooth library in block G (124). The information can be returned to the user device 108 for presentation in one or more GUIs. The information can be transmitted to a data store for storage and later retrieval. The information can be transmitted to another computing system of another relevant user, such as an oral surgeon.

In some implementations, the information can be fed back to the computer system 102 and used by other engines or components of the system 102 to auto design the dental appliance for the patient. For example, the information about the candidate tooth library can be transmitted to an engine configured to auto-adjust placement of teeth for the patient's smile and/or for their dentures design. Refer to at least the '217 application, incorporated herein by reference in its entirety, for further discussion about designing the dentures, and dental appliances more generally, using the candidate tooth library.

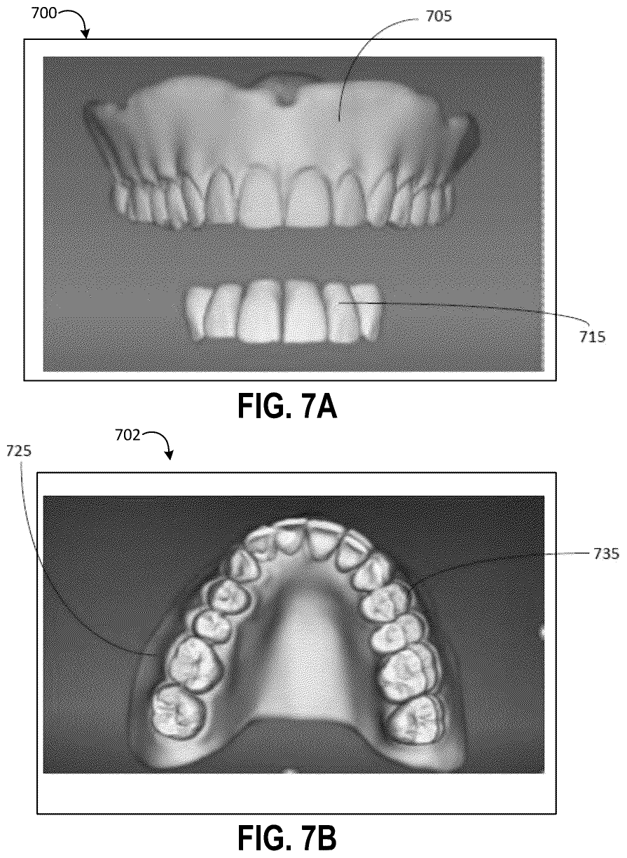

When the information is transmitted to the user device 108 in block G (124), the user device can output the information (block H, 126). The information can be outputted in GUIs, as shown, for example, in FIGS. 7-9. The candidate tooth library can be presented as overlaying at least a portion of a digital model of the dental appliance for the patient (or existing teeth of the patient). This visualization can help the relevant user identify how the teeth of the candidate tooth library would fit for the particular patient.

The relevant user can optionally design the dental appliance(s) for the patient using the selected candidate tooth library (block I, 128). As described herein and in the '217 application, the user can provide user input indicating one or more adjustments (e.g., movements) of the teeth presented in the GUIs. The user can also select options to perform auto-adjustments to the teeth presented in the GUIs. The auto-adjustments can be executed by the computer system 102 in order to automatically design the dental appliance(s) for the patient.

Although blocks A-G (112-124) are described with respect to the tooth library selection engine 104, one or more of the blocks can also be performed by other components of the computer system 102, as described further in reference to FIG. 10.

FIG. 1B illustrates an example digital dental model 160 with a selected tooth library 162 for a tooth implant. As described herein, the tooth library selection techniques can be used to select tooth libraries for dentures, smiles, and other types of dental appliances, such as implants and inserts. FIG. 1B shows that once the computer system described herein automatically selects the tooth library 162 that best fits for the patient's implant, the computer system can visually depict the selected tooth library 162 in placement with other teeth in the digital dental model 160. The digital dental model 160 can then be presented/outputted in a GUI described herein at a user device of a relevant user, such as a technician. The user can view how the selected tooth library 162 would look and/or fit with the patient's existing teeth. The user can also provide user input at their user device to move and/or adjust the selected tooth library 162 in the digital dental model 160. In some implementations, the user can provide user input indicating rejection of the selected tooth library 162. The user can then select a different tooth library presented in the GUI and the computer system described herein can update the digital dental model 160 to include the user-selected tooth library.

FIG. 1C is a conceptual diagram of system components for selecting tooth libraries. The computer system 102 described herein can include the tooth library selection engine 104, an auto-design denture setup engine 1209, a perfect smile setup engine 1209, and/or an auto-design implant setup engine 1211. The computer system 102 can include additional or fewer engines.

The tooth library selection engine 104 can communicate (e.g., wired and/or wirelessly via networks described herein) with one or more of the engines 1208, 1209, and 1211. In some implementations, the engine 104 may communicate with only one of the engines 1208, 1209, and 1211. The engine 104 can communicate with any of the engines 1208, 1209, and 1211 at same or different times.

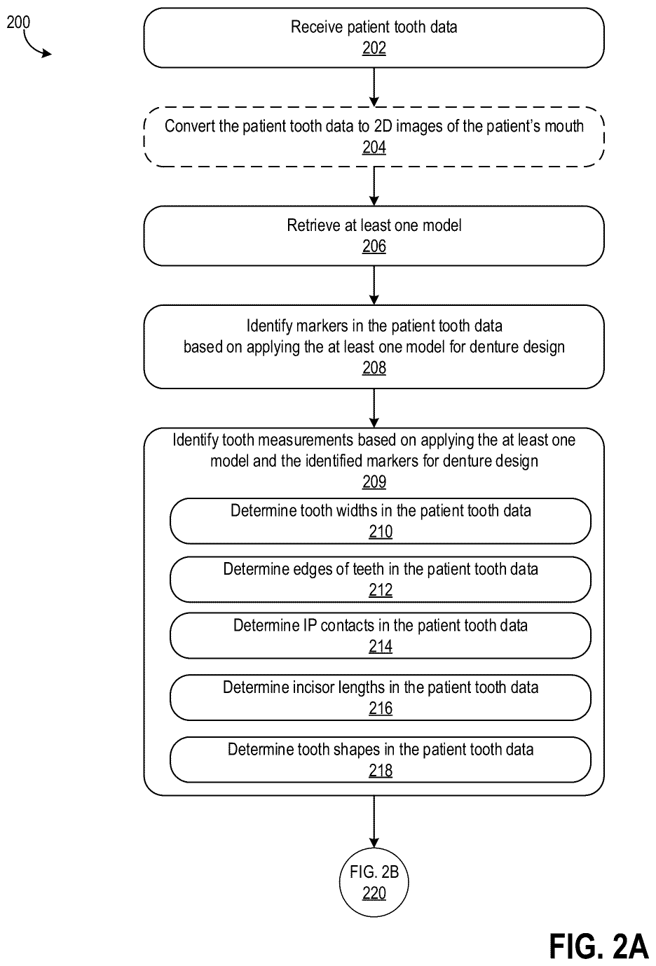

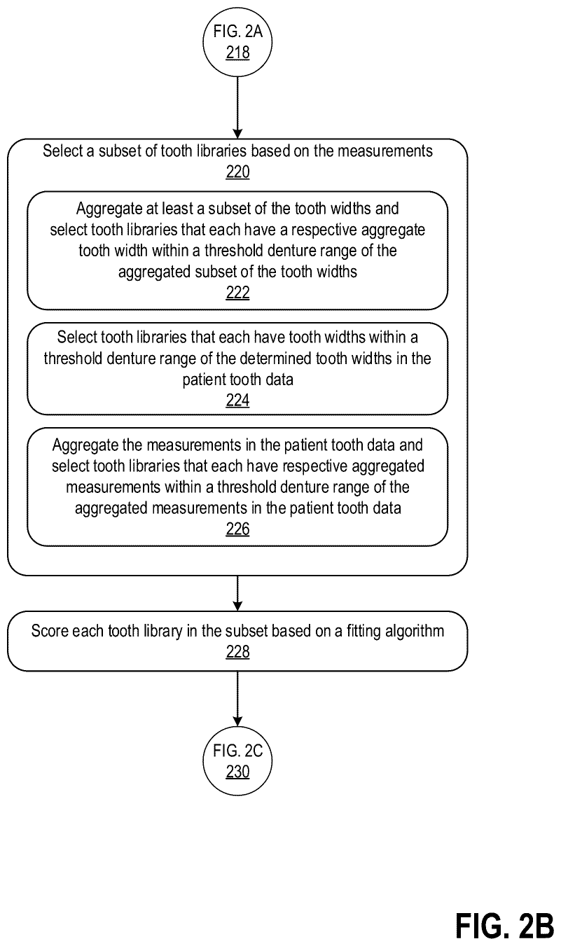

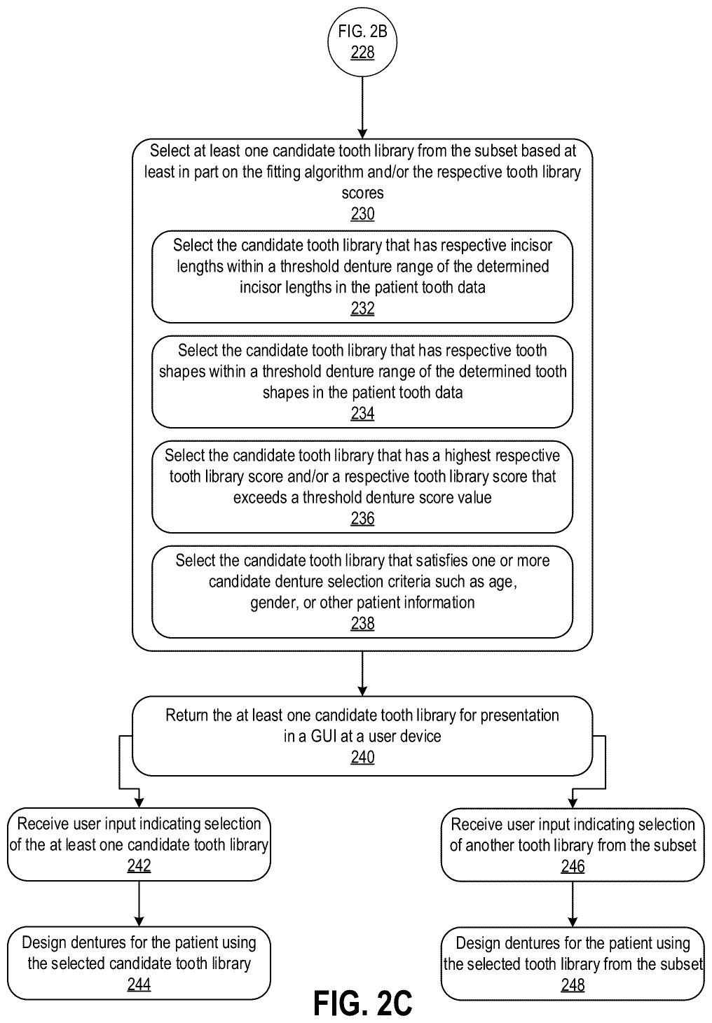

A an example, the auto-design denture setup engine 108 can transmit a request (block A, 150) to the tooth library selection engine 104 for a subset of tooth libraries and/or at least one candidate tooth library to be used in designing dentures for a particular patient. Upon receiving the request, the tooth library selection engine 104 can perform the techniques described herein, such as described in reference to FIGS. 2A, 2B, and 2C, to select the tooth library or libraries. For example, the engine 104 can determine aggregated tooth measurements for the particular patient (e.g., aggregated or combined tooth widths) and then select the tooth library having respective aggregated tooth measurements that are within a threshold range of or closest to the aggregated tooth measurements for the particular patient. The engine 104 can then transmit the selected tooth library or libraries to the auto-design denture setup engine 1208 (block B, 152). The engine 1208 can then auto-design the dentures for the particular patient using the selected tooth library or libraries.

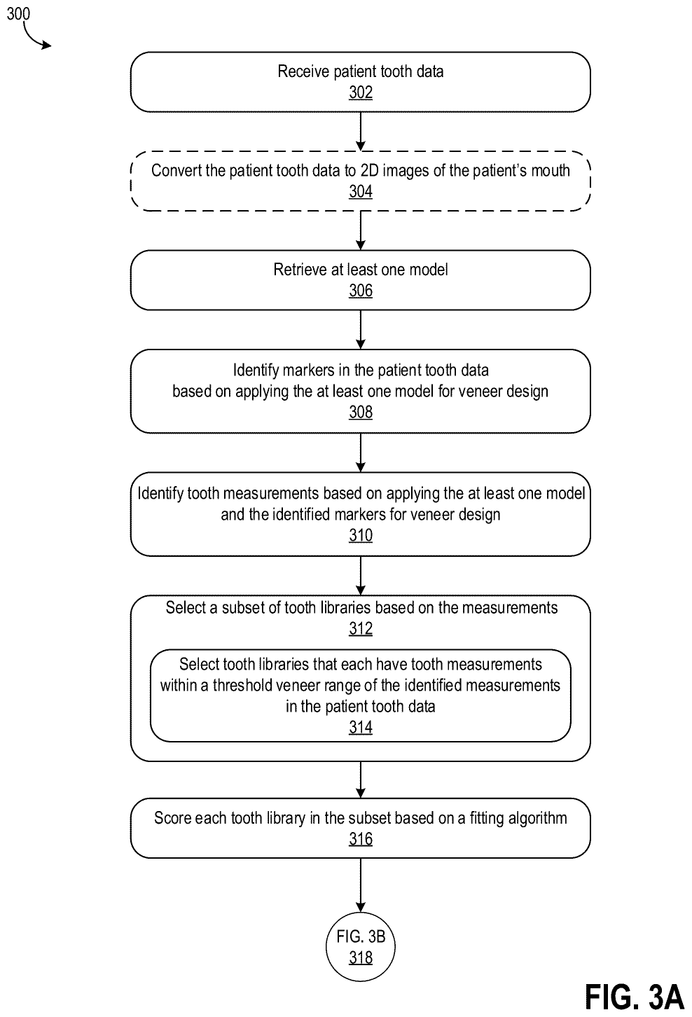

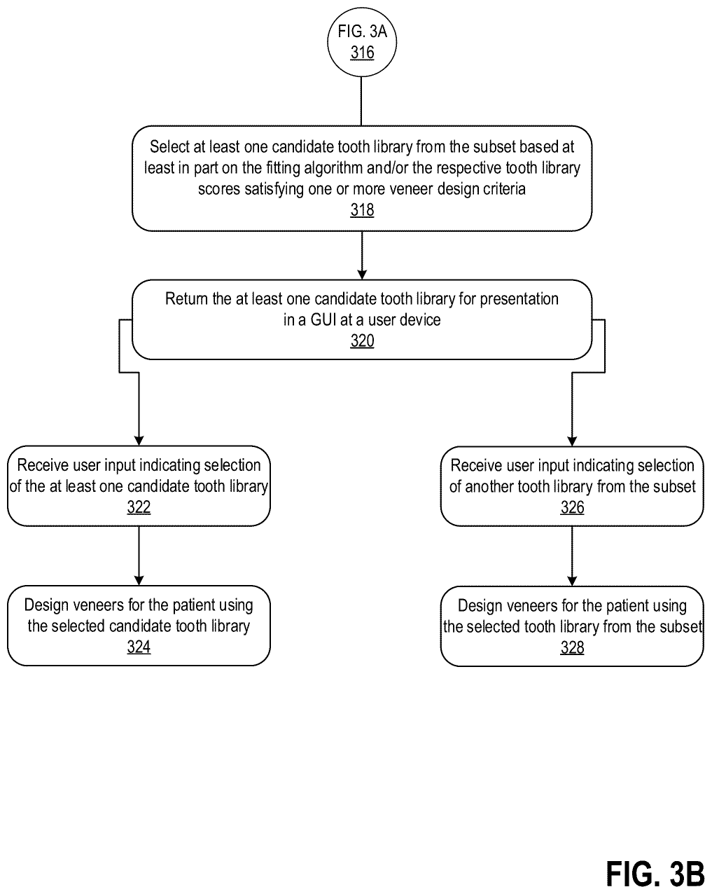

As another example, the perfect smile setup engine 1209 can transmit a request (block A, 150) to the tooth library selection engine 104 for a subset of tooth libraries and/or at least one candidate tooth library to be used in designing veneers for a particular patient (or other dental appliances that may be used for improving the patient's smile, including but not limited to orthodontics). Upon receiving the request, the tooth library selection engine 104 can perform the techniques described herein, such as described in reference to FIGS. 3A and 3B to select the tooth library or libraries. The engine 104 can use different selection criteria for the perfect smile setup engine 1209 request versus the auto-design denture setup engine 1208 request. For example, the engine 104 can compare aggregated tooth widths for tooth libraries with the patient's teeth. Designing veneers may have smaller degrees of freedom regarding movement and position than designing dentures because the veneers have to fit on an outer, facial, or front surface of the patient's teeth and without negatively impacting the patient's appearance. Therefore, the engine 104 can select tooth libraries with aggregated tooth widths that satisfy smaller threshold levels or ranges of acceptable tooth width for veneers design than for dentures design. The engine 104 can also use other criteria to select the tooth libraries for the perfect smile setup engine 1209. The engine 104 can then transmit the selected tooth library or libraries to the perfect smile setup engine 1209 (block B, 152). The engine 1209 can then auto-design the veneers and/or smile for the particular patient using the selected tooth library or libraries.

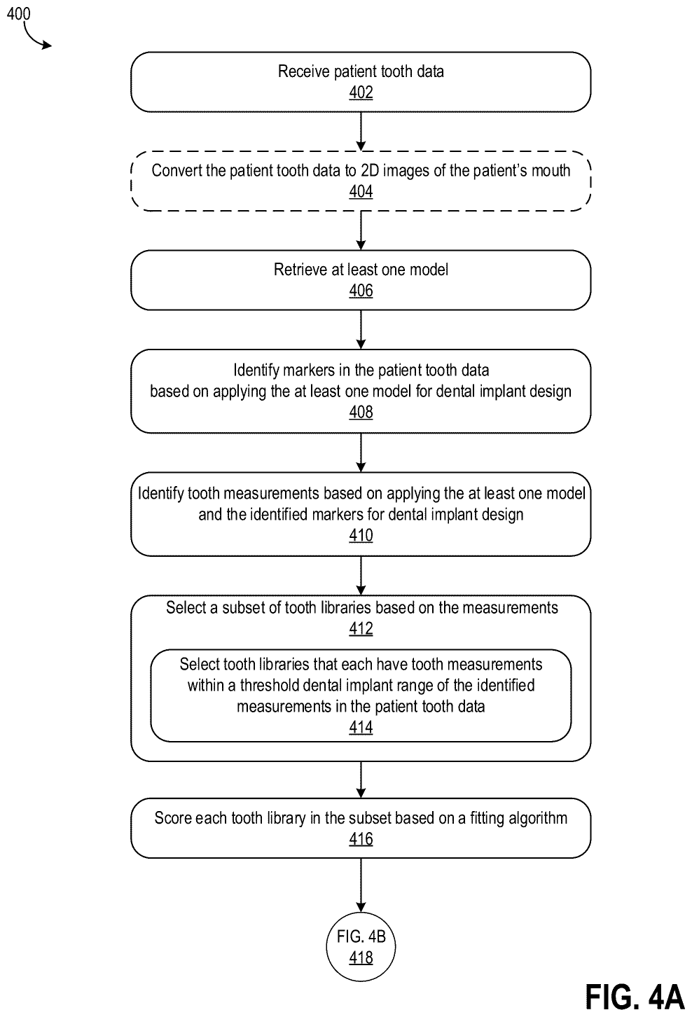

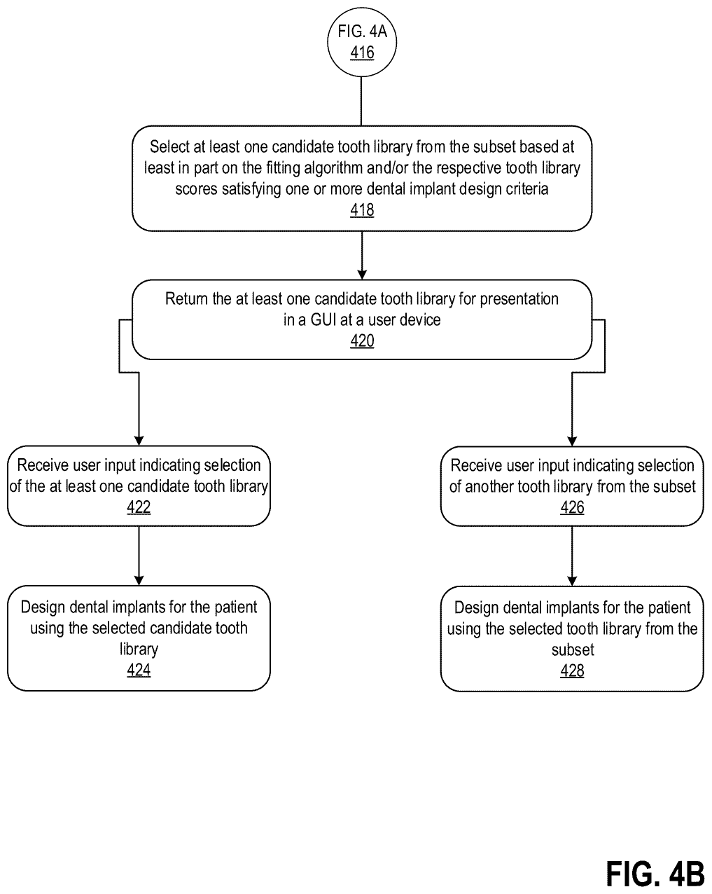

As yet another example, the auto-design implant setup engine 1211 can transmit a request (block A, 150) to the tooth library selection engine 104 for a subset of tooth libraries and/or at least one candidate tooth library to be used in designing dental implants or inserts (e.g., crowns, caps) for a particular patient. Upon receiving the request, the tooth library selection engine 104 can perform the techniques described herein, such as described in reference to FIGS. 4A and 4B to select the tooth library or libraries. The engine 104 can use different selection criteria for the auto-design implant setup engine 1211 request versus the auto-design denture setup engine 1208 request and/or the perfect smile setup engine 1209 request. For example, the engine 104 can compare aggregated tooth widths for tooth libraries with the patient's teeth. Designing implants and inserts may have smaller degrees of freedom regarding movement and position than designing dentures or even veneers because the implants or inserts have to fit into surface area of the patient's existing teeth and/or bone in the patient's upper and/or lower jaws. Therefore, the engine 104 can select tooth libraries with aggregated tooth widths that satisfy smaller threshold levels or ranges of acceptable tooth width for implant or insert design than for dentures or veneers design. The engine 104 can also use other criteria to select the tooth libraries for the auto-design implant setup engine 1211. The engine 104 can then transmit the selected tooth library or libraries to the auto-design implant setup engine 1211 (block B, 152). The engine 1211 can then auto-design the dental implants or inserts for the particular patient using the selected tooth library or libraries.

FIGS. 2A, 2B, and 2C are a flowchart of a process 200 for selecting tooth libraries for designing a denture for a patient. The process 200 can be performed by the computer system 102. More specifically, the process 200 can be performed by the tooth library selection engine 104 of the computer system 102. The process 200 can also be performed by any other engine or computing system described herein. For illustrative purposes, the process 200 is described from the perspective of a computer system.

Referring to the process 200 in FIGS. 2A, 2B, and 2C, the computer system can receive patient tooth data in block 202. Refer to block A (112) in FIG. 1A for further discussion about the patient tooth data. The patient tooth data can include 3D scans, 3D image data, or other types of scans or imaging of the patient's face, smile, mouth, jaw, etc. In some implementations, the patient tooth data can include jaw movement, patient teeth measurements, and/or information about the patient's dental appliances, molds, designs, demographics, etc. Sometimes, the patient tooth data can include 2D images of the patient's face, smile, mouth, jaw, etc.

The computer system can optionally convert the patient tooth data to 2D images of the patient's mouth in block 204. For example, if the patient tooth data includes 3D scans of the patient's mouth, then the computer system can apply techniques to convert the 3D scans to 2D image data, such as rendering an image of the 3D scan and capturing that image in 2D.

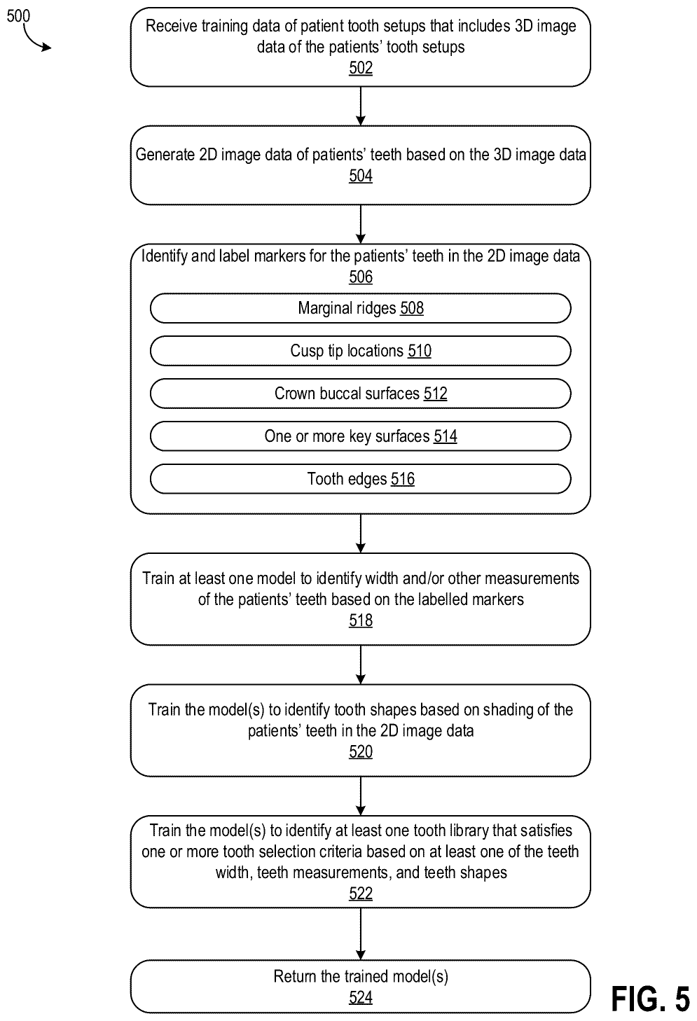

In block 206, the computer system can retrieve at least one model. The model can retrieved from a data store. The model can be a machine learning model trained as described in reference to FIG. 5. The model can be trained and used to identify markers or other landmarks in the patient tooth data. Additionally or alternatively, the model can be trained to measure each tooth and/or groups of teeth in the patient tooth data based on the identified markers. Additionally or alternatively, the model can be trained to identify and select predefined tooth libraries from a data store that have measurements within predetermined ranges or thresholds of the measurements of the tooth/teeth in the patient tooth data. Additionally or alternatively, the model can be trained to score each selected tooth library (e.g., using a fitting algorithm). Additionally or alternatively, the model can be trained to select a candidate tooth library for use in designing the patient's dental appliance(s) (e.g., dentures). The model can be trained to perform any of the abovementioned operations. In some implementations, multiple models can be retrieved in block 206, and each model can be trained to perform a different operation or set of operations.

The computer system can identify markers in the patient tooth data based on applying the at least one model for denture design in block 208. The model can be trained to identify different types of markers or landmarks per tooth type. For example, the model can be trained to identify cusp tip locations on incisors. Using the markers for the cusp tip locations, the computer system can then measure a length of the incisors. The length measurement can be used by the computer system to select a tooth library to be used for designing the patient's dentures or other dental appliances. The model can also be trained to identify opposing edges of each of the patient's teeth in the patient tooth data. Markers for the opposing edges of a tooth can then be used by the computer system to measure a width of the tooth, as described further below. In some implementations, the model can be trained to identify contacts between one or more teeth in the patient tooth data. For example, the model can identify proximal contacts (e.g., neighboring or adjacent teeth that come into contact), which can be used to determine individual tooth widths and/or an aggregate teeth width for a set or group of teeth. As another example, the model can be trained to identify an incisal edge and a point where a crown of the tooth starts to meet a root of the tooth. The computer system can use these markers to measure a crown height for upper central incisors—from a facial view, the computer system can measure the crown height as a distance from the incisal edge up to where the crown starts to blend into the root. This measurement can then be used to select a tooth library for the patient.

The computer system can identify tooth measurements based on applying the at least one model and based on the identified markers for denture design (block 209). In some implementations, the computer system can use AI algorithms that have been developed to determine measurements using the markers that were previously identified. Additionally or alternatively, the computer system can identify edentulous space measurements (e.g., cavity measurements) where the patient is missing a tooth. The edentulous space measurements can be used similarly to the tooth measurements described herein.

As an illustrative example, the computer system can determine tooth widths in the patient tooth data (block 210). The computer system can use the identified markers for opposing edges of a tooth to measure a distance between the opposing edges. The distance can be a tooth width. In some implementations, the computer system can identify markers for (i) a first edge on a first tooth and (ii) a second edge on a second tooth along an arch curve and/or in a set of teeth. The computer system can then measure a total distance between the first and second edges to determine a total combined width for the set of teeth. In some implementations, the computer system can measure from a gum/root to cusp tips of one or more of the teeth. In yet some implementations, the computer system can measure diagonally across a face of one or more of the teeth.

As another example, the computer system can determine edges of teeth in the patient tooth data (block 212). Sometimes, the edges can be determined as part of identifying the markers in block 208. Sometimes, the identified markers can be used in block 212 to identify the edges of each tooth.

As another example, the computer system can determine interproximal (IP) contacts in the patient tooth data (block 214). Thus, the computer system can identify which parts of a tooth contact (e.g., mesially and/or distally) an adjacent tooth in a same arch. Such contacts can be used by the computer system in determining the measurements of the tooth, such as a width of the tooth and/or a total width of multiple teeth that are in contact along the same arch form/curve. The computer system can use an AI algorithm and/or machine learning model to identify the IP contacts for a particular tooth and/or a set of teeth. In some implementations, the IP contacts can be identified as part of identifying the markers in block 208.

As yet another example, the computer system can determine incisor lengths in the patient tooth data (block 216). The markers determined in block 208 can be used to measure a length of an incisor (e.g., from root to cusp tip, from gum contact to cusp tip).

As another example, the computer system can determine tooth shapes in the patient tooth data (block 218). The computer system can apply a machine learning model to the patient tooth data that was trained to learn geometry of each tooth and match the geometry of a tooth in the patient tooth data with a predetermined or expected tooth shape. Each type of tooth can have a library of predetermined or expected tooth shapes, which can be used by the computer system to determine the shape of the tooth in the patient tooth data. As described herein, the model can be trained to identify tooth shapes including but not limited to square, tapered, and/or ovoid. The computer system can determine which of the tooth libraries has similar or same tooth shapes as the identified tooth shapes.

Once the computer system determines the measurements in blocks 209-218, the computer system can select a subset of tooth libraries from a data store based on the determined measurements (block 220). The computer system can access a static tooth library data store containing predetermined/predefined tooth libraries. The computer system can identify and select tooth libraries from the data store that have similar tooth measurements (e.g., exact measurements, within a threshold value or range) to the measurements of the teeth that were determined in blocks 209-218. In some implementations, the computer system can select the subset of tooth libraries using a best fit algorithm, as described above.

For example, the computer system can aggregate at least a subset of the tooth widths and select tooth libraries that have respective aggregate tooth widths within a threshold denture range of the aggregated subset of the tooth widths of the patient tooth data (block 222). The computer system can determine the aggregate width for a set of teeth in an upper arch form of the patient tooth data. The computer system can then compare that aggregated width to aggregated widths of teeth in upper arch forms of predefined tooth libraries in the data store. The computer system can select the tooth libraries from the data store that have aggregated tooth widths that are the same as or within the threshold range of the aggregated width for the particular patient's set of teeth in the upper arch. The computer system can select the tooth libraries having the same, similar, or closest aggregated tooth widths as the aggregated width for the particular patient. The tooth libraries can include measurements such as total widths of anterior teeth (on one side of an arch and/or across a full arch), total widths of posterior teeth (on one side of an arch and/or across a full arch).

As indicated herein, the threshold range can be specific to denture design. The threshold range can be larger (e.g., thereby allowing for identification and selection of tooth libraries that deviate more from the aggregated width of the patient's set of teeth) than a threshold range for designing veneers or dental implants/inserts (because the veneers, inserts, and/or implants may have a smaller degree of freedom in movement compared to the dentures). In some implementations, the threshold range can be approximately 1 mm. Sometimes, the computer system can select the tooth library having a closest fit/similarity in aggregated tooth widths or one or more individual tooth widths, so long as the selected tooth library also has a similar or same tooth shape as the shape of the patient's teeth. In some implementations, the computer system can select all tooth libraries within the threshold range (e.g., within 1 mm) of the aggregated width for the particular patient and present those tooth libraries to the relevant user. The user can then select which of the tooth libraries to try for the patient.

As another example, the computer system can select one or more tooth libraries that each have tooth widths within a threshold denture range of the determined tooth widths in the patient tooth data (block 224). The block 224 can be performed similarly to the block 222 described above. The threshold denture range in block 224 can be specific to a threshold width for a single tooth (and/or a particular type of tooth), whereas the threshold denture range in block 222 can be specific to a threshold width for the particular set/group of teeth whose width measurements are being aggregated. Moreover, measuring tooth widths and selecting tooth libraries based on the tooth widths can be faster and more efficient than other techniques since measuring the widths can be less computationally intensive and thus use less computing resources/processing power for simple calculations.

As yet another example, the computer system can aggregate the measurements in the patient tooth data and select one or more tooth libraries that each have respective aggregated measurements within a threshold denture range of the aggregated measurements in the patient tooth data (block 226). Similar to the block 222, the computer system can aggregate similar measurements for a set of teeth along an arch form/curve, then compare the aggregated measurements for the patient's set of teeth to aggregated measurements for similar sets of teeth in tooth libraries of the data store.

The computer system can score each of the selected tooth libraries in the subset based on a fitting algorithm in block 228. The computer system can apply a model, in some implementations, that was trained to score the selected tooth libraries based on how similar the measurements of the selected tooth libraries are to the measurements of the patient's teeth. The more similar the measurements, the higher the assigned score. The less similar the measurements, the lower the assigned score. Scores can be assigned using various different scales. For example, the scores can be assigned as integers, numeric values, and/or string values. In some implementations, the scores can be assigned on scales including but not limited to 0 to 5, 0 to 10, 0 to 100, 1 to 5, 1 to 10, 1 to 100, etc.

In block 230, the computer system can select at least one candidate tooth library from the subset based at least in part on the fitting algorithm and/or the respective tooth library scores. The computer system can employ one or more AI algorithms or machine learning models to select the candidate tooth library from the subset. The computer system can also eliminate potential selections down to one selected tooth library that matches best to the measurements of the patient's teeth. The computer system can optionally use demographics or other patient information to narrow down selection of the candidate tooth library. For example, the computer system can remove or eliminate tooth libraries from the subset that are intended for a particular gender, age, or type of dental appliance that does not correspond to a gender, age, or type of dental appliance for the particular patient.

In some implementations, the computer system can perform an iterative process of eliminating tooth libraries from the subset. For example, the computer system can first eliminate tooth libraries from the subset having tooth width measurements that fall outside a predetermined threshold tooth range of the patient's tooth width measurements. If the remaining subset still contains multiple tooth libraries, the computer system can then eliminate tooth libraries having central incisor lengths that fall outside a predetermined threshold tooth range of the patient's central incisor length measurements. If the remaining subset still contains more than one tooth library, the computer system can eliminate tooth libraries having tooth shapes that fall outside of threshold expected tooth shapes for the particular patient. In some implementations, if the remaining subset still contains more than one tooth library, the computer system can select a tooth library having the highest score, then return that selected tooth library as the candidate tooth library. Additionally or alternatively, the computer system can return all of the remaining tooth libraries in the subset as candidate tooth libraries. A relevant user can then select one of the candidate tooth libraries for designing the dentures for the patient.

For example, the computer system can select the candidate tooth library that has respective incisor lengths within a threshold denture range of the determined incisor lengths in the patient tooth data (block 232). In some implementations, the computer system can use tooth width measurements to select the tooth libraries that are initially added to the subset of tooth libraries. Then, the computer system can perform the block 232 to narrow down the subset of tooth libraries that have similar widths to the patient's teeth to the candidate tooth library. Thus, incisor lengths can be used to identify and select the candidate tooth library for the patient.

As another example, the computer system can select the candidate tooth library that has respective tooth shapes within a threshold denture range of the determined tooth shapes in the patient tooth data (block 234). As mentioned above, the computer system can use the tooth width measurements to select the tooth libraries initially added to the subset. Then, the computer system can perform the block 232 or the block 234 to narrow down the subset having the similar tooth widths to select the candidate tooth library. In some implementations, the computer system can perform the block 232 and then the block 234 to select the candidate tooth library (if the candidate tooth library is not selected after performing the block 232). In some implementations, the computer system can perform the block 234 and then the block 232 to select the candidate tooth library (if the candidate tooth library is not selected after performing the block 234).

As another example, the computer system can select the candidate tooth library that has a highest respective tooth library score and/or a respective tooth library score that exceeds a threshold denture score value (block 236). As mentioned above, the block 236 can be performed after performing either of the blocks 232 and 234, assuming that the candidate tooth library was not selected after performing either of those blocks. In some implementations, only the block 236 can be performed to select the candidate tooth library and/or the block 236 can be performed in combination with any one or more of the blocks 232 and 234. In yet some implementations, the blocks 232, 234, and 236 can be performed in any other order until the candidate tooth library is selected or the subset only contains one tooth library, which can be identified as the candidate tooth library.

As yet another example, the computer system can select the candidate tooth library that satisfies one or more candidate denture selection criteria, such as age, gender, or other patient information (block 238). As mentioned above, the block 238 can be performed after performing any of the blocks 232, 234, and 236, assuming that the candidate tooth library was not selected after performing one or more of those blocks. In some implementations, only the block 238 can be performed to select the candidate tooth library, and/or the block 238 can be performed in combination with any one or more of the blocks 232, 234, and 236. In yet some implementations, the blocks 232, 234, 236, and 238 can be performed in any other order until the candidate tooth library is selected or the subset only contains one tooth library, which can be identified as the candidate tooth library.

In blocks 230-238, the computer system can select one candidate tooth library. In some implementations, the computer system can select multiple candidate tooth libraries (e.g., tooth libraries having respective scores that exceed the threshold denture score value, a top 3 highest-scoring tooth libraries, tooth libraries having same high or highest scores, etc.).

The operations described with respect to blocks 230-238 can be performed in one or more other orders. For example, the computer system can start with determining a shape of the patient's teeth, then select the candidate tooth library, or a set of candidate tooth libraries, having the same or similar shape (block 234). Since the computer system can scale teeth to make them taller, shorter, wider, and/or narrower, identifying a best fitting tooth library based on tooth shape can be a first operation performed. Then, the computer system can narrow down a selection of the candidate tooth library by comparing tooth width measurements and/or tooth height measurements for the patient with those measurements of the tooth libraries.

In block 240, the computer system can return the at least one candidate tooth library for presentation in a GUI at a user device. In some implementations, the computer system can return the subset of the tooth libraries for presentation in the GUI. The user can then view the subset of select one of the subset as the candidate tooth library for designing the patient's dentures. In some implementations, the computer system can cause the candidate tooth library to be presented in the GUI, but the user can then select one of the other tooth libraries in the subset for presentation in the GUI and subsequent use in designing the patient's dentures.

The computer system can generate the GUIs for presentation at the user device. The GUIs can provide user-selectable and visual tools for the relevant user to view the patient tooth data and design (manually and/or automatically) dental appliances for the particular patient. Refer to at least FIGS. 7-9 and the '217 application for further discussion about the GUIs.

In some implementations, the process 200 can then stop. The process 200 can continue with blocks 242 and 244. The process 200 can additionally or alternatively continue with blocks 246-248.

For example, in blocks 242 and 244, the computer system can receive user input indicating selection of the at least one candidate tooth library (block 242) and then design dentures for the patient using the selected candidate tooth library (block 244). As described further in reference to at least the '217 application, the user can select an option (e.g., button) indicating approval of the candidate tooth library for use in designing the dentures for the patient. The computer system can then perform an auto-denture design process described further in the '217 application using the selected candidate tooth library. During this process, the computer system can iteratively adjust the selected candidate tooth library to fit a digital denture model for the patient, which can then be used to manufacture/fabricate dentures for the patient.

As another example, in blocks 246 and 248, the computer system can receive user input indicating selection of another tooth library from the subset (block 246) and then design dentures for the patient using the selected tooth library from the subset (block 248). As described above, the computer system can cause the user device to present the subset of tooth libraries in the GUI, or a portion of the tooth libraries in the subset (e.g., 3 highest-scored tooth libraries). The user can then view any of the presented tooth libraries and decide whether and which ones they desire to use in designing the dentures for the patient. The computer system can receive the user input indicating selection of the tooth library and then perform the auto-denture design process described in at least the '217 application to design the dentures for the patient.

In some implementations, the process 200 can begin once the computer system receives a request for tooth libraries to design dentures for the patient. The request can be received from the user device of the relevant user. In some implementations, the request can be received from an engine or other component of the computer system, such as an auto-design denture setup engine as described herein (e.g., refer to FIG. 1C and FIG. 10). In some implementations, the process 200 can be triggered by user input received at the user device or any other conditions being met as described throughout the disclosure.

FIGS. 3A and 3B are a flowchart of a process 300 for selecting tooth libraries for designing veneers for a patient. The process 300 can be performed to design veneers that can improve the patient's smile and/or appearance. The process 300 can be performed by the computer system 102. More specifically, the process 300 can be performed by the tooth library selection engine 104 of the computer system 102. The process 300 can also be performed by any other engine or computing system described herein. For illustrative purposes, the process 300 is described from the perspective of a computer system.

Referring to the process 300 in FIGS. 3A and 3B, the computer system can receive patient tooth data in block 302. For example, the patient's face can be scanned or imaged. Then, using AI algorithms, the computer system can identify eye sockets in the patient's face and measure the eye sockets. The computer system can also determine other geometry of the patient's face, which can be used to determine a preferred smile/teeth arrangement for the patient and to identify a tooth library for use in designing veneers for the patient. Refer to block 202 in the process 200 of FIGS. 2A, 2B, and 2C for further discussion.

Optionally, the computer system can convert the patient tooth data to 2D images of the patient's mouth in block 304. Refer to block 204 in the process 200 for further discussion.

In block 306, the computer system can retrieve at least one model. Refer to block 206 in the process 200 for further discussion.

The computer system can identify markers in the patient tooth data based on applying the at least one model for veneer design in block 308. Refer to block 208 in the process 200 for additional discussion. Sometimes, the markers identified in the process 300 can be different from the markers identified in the process 200. For designing veneers in the process 300, the computer system can identify additional or other markers, such as geometric lines between the patient's eyes, nose, mouth, cheeks, jaw, chin, and other facial features. Such geometric lines can be used to identify and select tooth libraries for veneer design that can improve symmetry, smile, and/or overall appearance of the patient's mouth and/or face.

The computer system can identify tooth measurements based on applying the at least one model and based on the identified markers for veneer design (block 310). Refer to block 209 in the process 200 for additional discussion about identifying measurements. In some implementations, since the computer system may identify markers that are unique or specific to veneer design, the computer system may determine measurements in block 310 that are specific to veneer design and the veneer-specific markers. In some implementations, tooth width, length, and/or shape can be used for selecting tooth libraries for veneer design, as described above for selecting tooth libraries for dentures design.

In block 312, the computer system can select a subset of tooth libraries from a data store based on the determined measurements. For example, the computer system can select tooth libraries that each have tooth measurements within a threshold veneer range of the identified measurements in the patient tooth data (block 314). Refer to blocks 220-226 in the process 200 for further discussion about selecting the subset of tooth libraries. The threshold ranges used for blocks 312 and 314 can be specific to veneer design, whereas the threshold ranges used in blocks 220-226 of the process 200 can be specific to denture design. As described throughout this disclosure, the threshold ranges for veneer design can be smaller than the threshold ranges for denture design because veneer design can have smaller degrees of freedom with respect to movement and size than denture design.

The computer system can score each of the selected tooth libraries in the subset based on a fitting algorithm (block 316). Refer to block 228 in the process 200 for additional discussion about scoring.

In block 318, the computer system can select at least one candidate tooth library from the subset based at least in part on the fitting algorithm and/or the respective tooth library scores satisfying one or more veneer design criteria. Refer to blocks 230-238 in the process 200 for additional discussion about selecting the candidate tooth library.

In block 320, the computer system can return the at least one candidate tooth library for presentation in a GUI at a user device. Refer to block 240 in the process 200 for additional discussion. In some implementations, the process 300 can then stop. In some implementations, the process 300 can continue with blocks 322 and 324. In other implementations, the process 300 can continue with blocks 326 and 328.

For example, in blocks 322 and 324, the computer system can receive user input indicating selection of the at least one candidate tooth library (block 322) and then design veneers for the patient using the selected candidate tooth library (block 324). Refer to blocks 242 and 244 in the process 200 for additional discussion.

As another example, in blocks 326 and 328, the computer system can receive user input indicating selection of another tooth library from the subset (block 326) and then design veneers for the patient using the selected tooth library from the subset (block 328). Refer to blocks 246 and 248 in the process 200 for additional discussion.

In some implementations, the process 300 can begin once the computer system receives a request for tooth libraries to design veneers, or a smile more generally for the patient. The request can be received from the user device of the relevant user. In some implementations, the request can be received from an engine or other component of the computer system, such as a perfect smile or auto-design veneers setup engine as described herein (e.g., refer to FIG. 1C and FIG. 10). In some implementations, the process 300 can be triggered by user input received at the user device or any other conditions being met as described throughout the disclosure.

FIGS. 4A and 4B are a flowchart of a process 400 for selecting tooth libraries for designing dental implants for a patient (e.g., dental inserts, crowns, caps). The process 400 can be performed by the computer system 102. More specifically, the process 400 can be performed by the tooth library selection engine 104 of the computer system 102. The process 400 can also be performed by any other engine or computing system described herein. For illustrative purposes, the process 400 is described from the perspective of a computer system.

Referring to the process 400 in FIGS. 4A and 4B, the computer system can receive patient tooth data in block 402. Refer to block 202 in the process 200 of FIGS. 2A, 2B, and 2C for further discussion.

Optionally, the computer system can convert the patient tooth data to 2D images of the patient's mouth in block 404. Refer to block 204 in the process 200 for further discussion.

In block 406, the computer system can retrieve at least one model. Refer to block 206 in the process 200 for further discussion.

The computer system can identify markers in the patient tooth data based on applying the at least one model for dental implant design in block 408. Refer to block 208 in the process 200 for additional discussion. In some implementations, as discussed with reference to block 308 in the process 300, the markers identified for dental implant design can be different than the markers identified for dentures and/or veneers. In some implementations, the markers identified for dental implant design can be the same as the markers identified for dentures and veneers. Similarly, tooth width, cavity, and/or shape information can be used for selecting a set of tooth libraries for the purposes of designing implants.

The computer system can identify tooth measurements based on applying the at least one model and based on the identified markers for dental implant design (block 410). Refer to block 209 in the process 200 for additional discussion.

In block 412, the computer system can select a subset of tooth libraries from a data store based on the determined measurements. For example, the computer system can select tooth libraries that each have tooth measurements within a threshold dental implant range of the identified measurements in the patient tooth data (block 414). Refer to blocks 220-226 in the process 200 for further discussion about selecting the subset of tooth libraries. As described in reference to the process 300, the threshold ranges used in block 412 and 414 can be different than the threshold ranges used in the processes 200 and 300. Dental implants, similar to veneers, can have a smaller degree of freedom with respect to movement and design. Therefore, the threshold dental implant ranges can be smaller than the threshold denture ranges described in the process 200. The threshold dental implant ranges can be similar, in some implementations, to the threshold veneer ranges of the process 300.

The computer system can score each of the selected tooth libraries in the subset based on a fitting algorithm (block 416). Refer to block 228 in the process 200 for additional discussion about scoring.

In block 418, the computer system can select at least one candidate tooth library from the subset based at least in part on the fitting algorithm and/or the respective tooth library scores satisfying one or more dental implant design criteria. Refer to blocks 230-238 in the process 200 for additional discussion about selecting the candidate tooth library.

In block 420, the computer system can return the at least one candidate tooth library for presentation in a GUI at a user device. Refer to block 240 in the process 200 for additional discussion. In some implementations, the process 400 can then stop. In some implementations, the process 400 can continue with blocks 422 and 424. In other implementations, the process 400 can continue with blocks 426 and 428.

For example, in blocks 422 and 424, the computer system can receive user input indicating selection of the at least one candidate tooth library (block 422) and then design dental implants for the patient using the selected candidate tooth library (block 424). Refer to blocks 242 and 244 in the process 200 for additional discussion.

As another example, in blocks 426 and 428, the computer system can receive user input indicating selection of another tooth library from the subset (block 426) and then design dental implants for the patient using the selected tooth library from the subset (block 428). Refer to blocks 246 and 248 in the process 200 for additional discussion

In some implementations, the process 400 can begin once the computer system receives a request for tooth libraries to design dental implants for the patient. The request can be received from the user device of the relevant user. In some implementations, the request can be received from an engine or other component of the computer system, such as an auto-design dental implant setup engine as described herein (e.g., refer to FIG. 1C and FIG. 10). In some implementations, the process 400 can be triggered by user input received at the user device or any other conditions being met as described throughout the disclosure.

FIG. 5 is a flowchart of a process 500 for training at least one model to be used in selecting tooth libraries for designing dental appliances for patients. The process 500 can be performed to generate the models described herein. The process 500 can also be used to iteratively train and/or improve the models described herein. In some implementations, the process 500 can be performed to train models per operation and/or dental appliance procedure. For example, a model can be trained to generally identify markers in patient tooth data. The same model or another model can be trained to determine tooth and/or cavity measurements based on the identified markers in the patient tooth data. The same or another model can be trained to score tooth libraries. The same or another model can be trained to select tooth libraries as possible candidate tooth libraries for any type of dental procedure. In some implementations, a first model can be trained to identify markers, determine measurements, and score tooth libraries for use in denture design. A second model can be trained to identify markers, determine measurements, and score tooth libraries for use in veneers design. A third model can be trained to identify markers, determine measurements, and score tooth libraries for use in dental implants/inserts design. One or more other models can be trained to perform any combination of the operations described herein.

Moreover, the models described herein can be trained on anterior and/or posterior teeth collections. For example, one model can be trained on only anterior teeth collections, another model can be trained on only posterior teeth collections, and yet another model can be trained on both anterior and posterior teeth collections. In yet some implementations, one or more of the models can be trained on thousands of training data, such as teeth selections and other patient teeth data. at least 50 separate teeth selections. The more training data used, the more accurate the models may be.

The process 500 can be performed by the computer system 102. The process 500 can also be performed by any other computing system described herein. For illustrative purposes, the process 500 is described from the perspective of a computer system. Moreover, although the process 500 is described as training models to perform a variety of operations, one or more models can be trained, using the process 500 or portions of the process 500, to perform less than all of the operations described herein.

Referring to the process 500 in FIG. 5, the computer system can receive training data of patient tooth setups that may include 3D image data of the patients' tooth setups (block 502). The training data can include any of the patient tooth data described herein. In some implementations, the training data can include raw images or scans of the patient's face, mouth, teeth, and/or jaw. The training data can include designs for the patient's dentures, veneers, brackets, crowns, caps, dental inserts, dental implants, or other dental appliances. The training data can include patient tooth data that has been annotated and/or labeled (e.g., manually by a relevant user such as a technician and/or automatically by the computer system). For example, the training data can include 2D images of the patient's teeth with labels or annotations indicating edges on each tooth, distances between edges on each tooth, shapes of each tooth, and other information that may be used to train the models described herein.

In block 504, the computer system can generate 2D image data of the patients' teeth based on the 3D image data. At least one model described herein can be trained on 2D images of the patient tooth data in order to accurately select tooth libraries for use in designing dental appliances for a particular patient. For example, some of the training data can include 3D libraries that are used, during runtime, to generate dental appliance setups. 2D images can be generated from the setups or 3D libraries, then used for model training.

The computer system can identify and label markers for the patients' teeth in the 2D image data (block 506). The computer or a relevant user can identify and label the markers. The computer system can identify and labels markers described throughout this disclosure that are generic to designing different types of dental appliances or specific to designing particular types of dental appliances. For example, as part of identifying and labeling the markers, the computer system can identify and label marginal ridges (block 508), cusp tip locations (block 510), crown buccal surfaces (block 512), one or more other key surfaces (block 514), and/or tooth edges (block 516). As described above, the patient tooth data may already be labeled and annotated, so block 506 may be skipped in the training process 500.

The computer system can train at least one model to identify width and/or other measurements of the patient's teeth based on the labelled markers (block 518). In some implementations, the model(s) can be trained using the training data that has already been labeled with widths and the other teeth measurements described throughout this disclosure (e.g., refer to the processes 200, 300, and 400 in FIGS. 2-4).

Additionally or alternatively, in block 520, the computer system can train the model(s) to identify tooth shapes based on shading of the patients' teeth in the 2D image data. For example, different shadows, colors, and/or shades of color in the 2D image data can be labeled/annotated as corresponding to different predefined tooth shapes. The model(s) can be trained with the labeled 2D image data to identify the different predefined tooth shapes in patient tooth data during runtime use.

Additionally or alternatively, the computer system can train the model(s) to identify at least one tooth library that satisfies one or more tooth selection criteria based on at least one of the tooth widths, tooth measurements, and/or tooth shapes (block 522). The training data can include associations that are predefined (e.g., automatically by the computer system and/or manually by relevant users) between labelled markers, measurements, and/or tooth shapes with different tooth libraries. The tooth libraries can be stored in a static data store as described herein and used for training as well as runtime use. The training data can also indicate which labelled markers, measurements, and/or tooth shapes satisfy one or more of the tooth selection criteria. Accordingly, the model(s) can be trained to determine when one or more of the tooth selection criteria are satisfied and which tooth library satisfies the criteria.

The computer system can then return the trained model(s) for runtime use in block 524. The model can be stored in a data store and then retrieved for later use (e.g., during runtime). The model can be stored in local memory/storage of the computer system for quick retrieval during runtime use. In some implementations, as described herein, multiple models can be trained. Some of the models, such as models that are trained to identify and label markers and/or determine tooth and/or cavity measurements can be stored locally at the computer system for quick and efficient processing during runtime. Other models, such as models that are trained to identify tooth libraries for dentures, dental implants/inserts, and/or veneers can be stored in the data store then retrieved during runtime, based on the type of dental appliance being designed. Thus, the computer system may only retrieve the model(s) specific to the type of dental appliance being designed, rather than locally storing all the models. This can provide for efficient use of available compute resources and processing power, which can improve efficiency of the computer system in selecting tooth libraries and designing the dental appliances.

As mentioned above, one or more blocks in the process 500 can also be performed as part of iteratively training the model(s). For example, during runtime, the model(s) can be used to select a candidate tooth library for designing dentures for a patient. The relevant user can view the candidate tooth library in a GUI presented at their user device and decide to select a different tooth library for use in designing the patient's dentures. The user's selection of the different tooth library can be transmitted to the computer system. The computer system can use the user's selection as training data for one or more of the models to improve the model(s) decision-making in selecting the candidate tooth library for designing dentures. As yet another example, the computer system can receive annotated patient tooth data from relevant users, such as technicians, dentists, orthodontists, etc., and use that data to iteratively improve and train the models described herein to more accurately identify markers, determine tooth measurements, score tooth libraries, and/or select tooth libraries for different dental procedures.

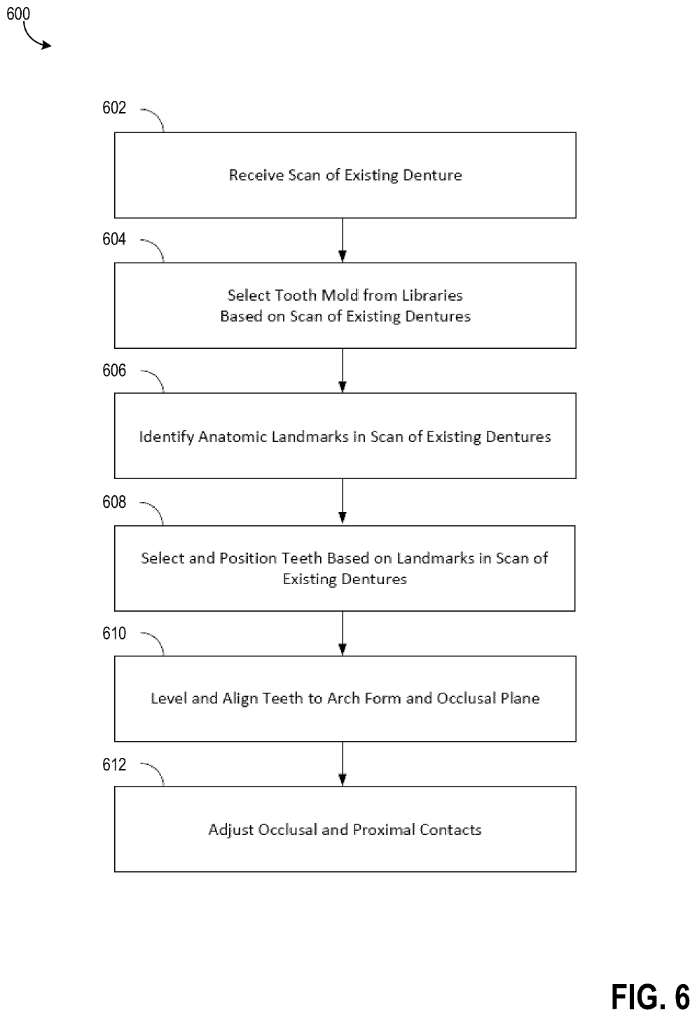

FIG. 6 is a flowchart of an example method 600 for automatic design of a replacement denture. Although the method 600 is described with respect to designing a replacement denture, the method 600 can also be performed to design other types of dental appliances, including but not limited to dentures, veneers, dental implants, dental inserts, caps, and/or crowns. The method 600 can be performed by the computer system 102 described herein. In some implementations, the method 600 can be performed by one or more other computing systems described throughout this disclosure. For illustrative purposes, the method 600 is described from the perspective of a computer system.

Referring to the method 600, at block 602, the computer system can receive a scan of an existing denture for a patient. In some implementations, the scan can be produced using a CT scanning device, a CBCT scanning device, an intraoral scanner, or a desktop scanner. In some implementations, the scan can be processed upon receipt by the computer system, for example by detecting and filling in voids in the scan, removing scan artifacts arising from metallic inclusions in the dentures, correcting holes in the scan arising from positioning of the dentures in a jig or on a surface during the scan, thresholding the scan data to produce a surface scan, and/or converting a file type of the scan to another file type. For example, in some implementations, the scan data can be converted from the native file type of the CT scanner (for example, a Digital Imaging and Communications in Medicine (DICOM) file) to a data model file type such as a stereolithography (STL) file type. In some implementations, the DICOM file or other native file of the CT scanner is converted to a polygon file format (PLY), a standard 3D image file format (OBJ), an additive manufacturing file format (AMF), a 3D manufacturing file format (3MF), or any other suitable file type.

Additional digital patient data can also be received, for example motion data, image data, and/or or color images of the dentures or patient's mouth. Motion data can be captured using a motion capture system and can represent the patient's jaw moving through various jaw movements for use in preparing a replacement denture design. Color images can be obtained simultaneously with the scanning of the existing dentures and can be used to overlay color on the denture design. Additional details related to the use of motion capture data and color images are described in U.S. Provisional Patent Application Ser. No. 63/149,178 filed on Feb. 12, 2021 and entitled “Motion-Based Digital Denture Design,” U.S. Provisional Patent Application Ser. No. 63/274,798 filed on Nov. 2, 2021 and entitled “Digital Denture Design and Replacement,” the PCT Application filed Feb. 10, 2022 and entitled “Digital Denture Design and Replacement,” and in U.S. Provisional Patent Application Ser. No. 63/313,723 filed on Feb. 24, 2022 and titled “Color Digital Denture Design and Replacement,” the contents of each of which are incorporated herein by reference in their entirety.

At block 604, a tooth mold (e.g., tooth library) can be selected by the computer system from a tooth library based on the scan of the existing dentures. Multiple tooth libraries exist that can include sets of teeth that can be chosen to include in dentures and other oral prosthetics. These libraries can be accessed and reviewed by the computer system to automatically select a tooth mold having a close fit (e.g., based on measurements such as tooth width, incisor length, and/or tooth shape) to the teeth of the existing denture. The digital denture teeth libraries may vary functionally, aesthetically, and/or based on manufacturer. The computer system can implement a number of mechanisms described throughout this disclosure for automatically selecting and scoring tooth libraries that can be used for the design process. The computer system can implement fitting algorithms and/or software scoring schemes to determine a best match and/or closest alternatives from the available tooth libraries or set of tooth libraries.

As described herein, the selection of the tooth libraries (e.g., tooth molds) can be made by the computer system based on automatic identification of landmarks and anatomical dimensions of a subset of teeth in the scan data. The selected tooth libraries can be applied to all teeth in the denture design, in some implementations. As an illustrative example, the width of a particular tooth can be used as an initial selection criteria for determining a best matching tooth library from the available sets of tooth libraries in a data store. In some implementations, the width of one or more teeth can be used to identify a subset of tooth libraries, and an iterative fitting algorithm or other machine learning models and/or AI algorithms can be used to determine a candidate tooth library from the subset of libraries. Such techniques can advantageously reduce an amount of processing power required for determining a best-fit tooth library compared to using an iterative algorithm across a large number of potential tooth libraries from which to select.

In some implementations, selection of the tooth library can be made for each individual tooth in the denture based on landmark and anatomical dimensions of the particular tooth. The selection of the tooth library can be made based on a tooth or a subset of teeth in an upper denture, and the selected tooth library that is a closest match can then be applied to the lower denture as well. In some implementations, selection of the tooth library can be independent for the upper denture and the lower denture. Sometimes, selection of the closest matching tooth library to the existing dentures can take into consideration landmarks and dimensions of teeth on both the upper and lower dentures. Additional information including, but not limited to, patient specific motion from motion capture scans, patient mouth/teeth images, and/or hinge axis information can also be used by the computer system to automatically match and select the candidate tooth library from available tooth libraries stored in the data store.

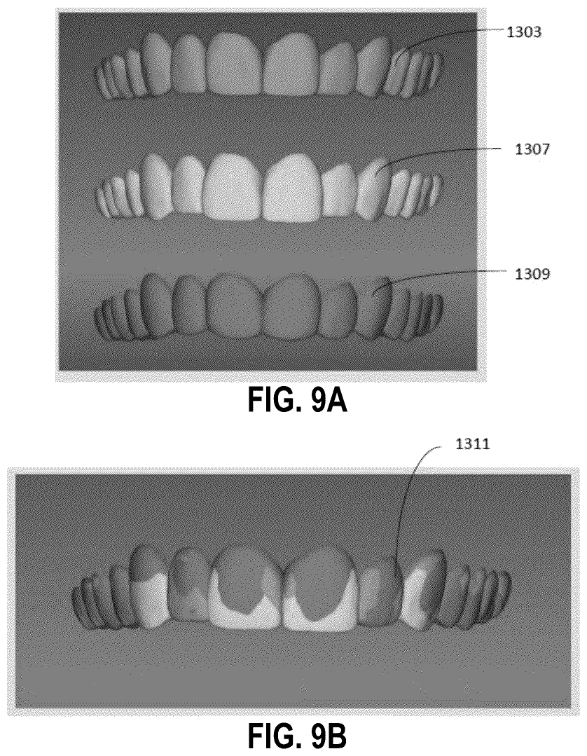

The selected tooth library may be displayed visually in one or more GUIs at a user device so that a relevant user (e.g., technician, dentist, orthodontist, oral surgeon) may confirm or reject the selection. The closest match can be automatically selected and presented in a GUI. Alternatively or additionally, a subset of closest tooth library matches can be presented in the GUI for user review and/or selection. In some implementations, several candidate tooth libraries (e.g., the three that have the closest width to that of the digital reference denture model) can be selected and presented to the user. In some implementations, the closest match can be presented with one, two, three, or four (or any other predetermined quantity) next closest matches for user confirmation and/or selection. The user may interact with the GUI to select between these options. In some implementations, the closest match and other next closest matches can be presented separately and/or in an overlay presentation using a color map to illustrate differences across the different tooth libraries. Refer to FIGS. 9A and 9B for further discussion.

Enabling the automatic matching of denture landmarks to an existing tooth library can allow for the computer system to efficiently identify closest matching teeth to an existing denture. The speed with which computer system can identify a match using an iterative fitting algorithm, such as iterative closest point (ICP) or another algorithm, machine learning model, and/or AI algorithm can be much greater than a speed with which the user can manually and visually determine an appropriate tooth library for the particular patient.

At block 606, the computer system can automatically identify anatomic landmarks in the scan of existing dentures (or other patient tooth data described herein). The identification of individual teeth can be determined by the computer system using one or more machine learning models that have been trained to identify and label/annotate different types of teeth. For example, the model(s) can be trained to identify and label molars, pre-molars, canines, and/or incisors. Additionally or alternatively, the width of each tooth, of particular teeth, and/or of groups of teeth can be determined by the computer system. The height, circumference, and/or cross-section can also be determined by the computer system. Other landmarks such as cusps, pits, offsets, and grooves can be identified by the computer system. The computer system can make any of these determinations using machine learning models and/or AI algorithms as described herein. The identified landmarks can be presented to the user through the GUIs described herein.

At block 608, the teeth from the selected tooth library can be selected and positioned on the denture scan by the computer system, and based on the identified landmarks and other tooth morphology. For example, a tooth from the tooth library can be selected for a particular tooth of the denture scan based on a shape matching of the teeth and/or based on a labeling of the tooth library and labeling applied to the scan using the identified landmarks. In some implementations, the digital denture teeth can be positioned based on a determined or selected occlusal guidance surface. In some implementations, the digital denture teeth may include labels for anatomical landmarks such as cusps, marginal ridges, incisal edges, fossa, grooves, base boundaries, or other anatomical landmarks. These labels may be used by the computer system to automatically position the digital denture teeth with respect to one another and digital denture teeth on opposing dentition.

For example, the digital denture teeth may be initially positioned in alignment with an arch curve. The arch curve may be sized and shaped, by the computer system and/or via user input at the GUIs, based on the digital dental model. Each of the digital denture teeth may include one or more labels that specify one or more locations on the digital denture teeth to align to the arch curve. The digital denture teeth may also be associated with a tip and torque with respect to one or more of the arch curve and the occlusal plane. When initially positioned, the digital denture teeth may be positioned based with respect to the arch curve based on the labels and automatically tipped and torqued with respect to the arch curve based on the associated values

Once the digital denture teeth are in their initial positions, their positions may be further refined. For example, at block 610, the teeth can be leveled and aligned to the arch form and to the occlusal plane by the computer system. Once selected, the tooth from the library tooth mold can be automatically positioned in the scan so as to most closely match the tooth in the existing denture. This can include leveling the teeth and aligning the teeth on the arch. For example, an ICP algorithm can be used to determine the appropriate position of the library tooth mold tooth to match the tooth in the existing denture. An ICP algorithm can determine a general “best-fit” and/or a best-fit within microns.

After the teeth are automatically adjusted to fit the arch and the digital scan, and the denture scan including the tooth library can also be presented in the GUI to enable the relevant user to manipulate and adjust the teeth. The computer system can automatically adjust the occlusal and proximal contacts in block 612. The GUI may also receive user input to move the selected digital denture tooth. In some implementations, the user input can include a drag input, such as a click-and-drag and/or touch-and-drag. Based on the direction of the drag, the digital denture tooth may move in a corresponding direction. In some implementations, the movement may be in a direction that is parallel to the occlusal plane. In some implementations, as the digital denture tooth moves based on the drag input, the digital denture tooth also moves in the occlusal-gingival direction to make contact with the opposing dentition. In some implementations, the digital denture tooth may move, by the computer system, to contact with an occlusal guidance surface that is generated based on the opposing denture teeth and the motion data (e.g., by sweeping the opposing denture teeth through the motion of the motion data).

Following block 612, the denture design can be finalized. A denture base can be automatically generated and presented in the GUI for approval and/or adjustment. In some implementations, a soft-tissue boundary curve can be generated by the computer system and based on the digital dental model. The soft-tissue boundary curve can represent an edge of the denture base. Once the soft-tissue interface is adjusted b the user and/or automatically by the computer system, tooth boundary curves can be identified for each of the positioned digital denture teeth. In some implementations, the denture design can be presented in the GUI for approval and then to fabricate the replacement dentures. For further discussion about auto-design of dentures, veneers, implants, inserts, and other types of dental appliances, refer to the '217 application, the disclosure of which is incorporated by reference in its entirety.

As described throughout this disclosure, automation of the denture design process (and the dental appliance design process more generally) can increase efficiency of denture design by reducing design aspects that a user must complete from scratch and/or manually. Automatically selecting tooth libraries, identifying and annotating landmarks, and positioning individual and/or groups of teeth can reduce an amount of work, time, and computing resources to accelerate the design process and reduce associated costs. Rather than begin the design from scratch and prepare the denture design based on visual inspection of existing dentures or scans, the computer system can automatically make suggestions based on advanced and complex best-fit algorithms to produce a design that closely matches the existing dentures in fit, function, and appearance. The high-quality denture designs generated using the automation of design processes described herein can reduce the time to design and produce dentures and reduce the cost of developing the denture design while minimizing differences in dentures designed by various technicians for improved patient experience.

FIGS. 7A and 7B illustrate example user interface screens (e.g., GUIs) 700 and 702 that may be generated during a denture design process of positioning selected tooth library teeth in a digital denture model and automatically aligning and leveling each of the selected tooth library teeth. Although the GUIs 700 and 702 are illustrate the denture design process, similar GUIs can be generated and presented for other design processes, including but not limited to veneers design, dental implants design, dental inserts design, etc.

The GUI 700 in FIG. 7A shows a digital denture model 705 and selected tooth model 715. The selected tooth model 715 can be a tooth library selected by the computer system using the techniques described herein. The selected tooth model 715 can be automatically positioned on the digital denture scan 705 by the computer system to align each tooth with a determined arch. Each tooth can also be automatically positioned such that the tooth is in a position that most closely matches teeth of the digital denture. In some implementations, an ICP algorithm can be used by the computer system to determine a best fit. The application of the ICP algorithm can be displayed in the GUI 700 to illustrate to a technician or other relevant user positioning of the teeth and degree of difference that remains when the teeth are positioned using the algorithm.

In some implementations, an overlay of the tooth library and the digital model from the denture scan can be displayed in the GUI with a color map or heat map to illustrate differences between the tooth library and the existing scan, as shown in FIGS. 9A and 9B. The colors of the color map can indicate varying degrees of difference between the tooth library and the existing scan. For example, a green color can indicate that the tooth library and the existing scan are very close in position, size, orientation, dimensions, etc. A yellow color can indicate that the positioning of the tooth library with the existing scan can be improved (by at least some threshold amount). A red color can indicate a large degree of difference between positioning of the tooth library and the existing scan. One or more other colors can be used to show differences between the tooth library and the existing scan when the tooth library overlays the existing scan in the GUIs described herein. One or more other indicia can also be used to show the differences, including but not limited to different patterns and/or textures.

The GUI 702 in FIG. 7B shows how the teeth of the selected tooth model 715 are positioned in the digital denture model 705 in a best-fit position 725. Differences between the teeth of the digital denture model 705 and the teeth of the selected tooth model 715 can be illustrated to the user by a heat map or color map. For example, the teeth of the digital denture model 705 can be presented in a first color or indicia (e.g., a green color) and the teeth of the selected tooth model 715 can be presented in a second color or indicia (e.g., a white color). This information may be beneficial to help the relevant user identify wear patterns and areas of the selected tooth model 715 that may need to be modified in the replacement denture to provide a better fit for the particular user.

In FIG. 7B, areas where the teeth of the selected tooth model 715 extend beyond boundaries of the teeth of the digital denture model 705 can be presented in another color or indicia, such as a color green (e.g., refer to position 735). The technician can use such a display to make further adjustments to the digital denture model teeth. The computer system described herein can also use this information to automatically make adjustments to the digital denture model. In yet some implementations, the relevant user can use the GUIs 700 and/or 702 in FIGS. 7A and 7B to understand changes in tooth shape from the patient's chewing or grinding habits, and make recommendations to the patient based on the information.

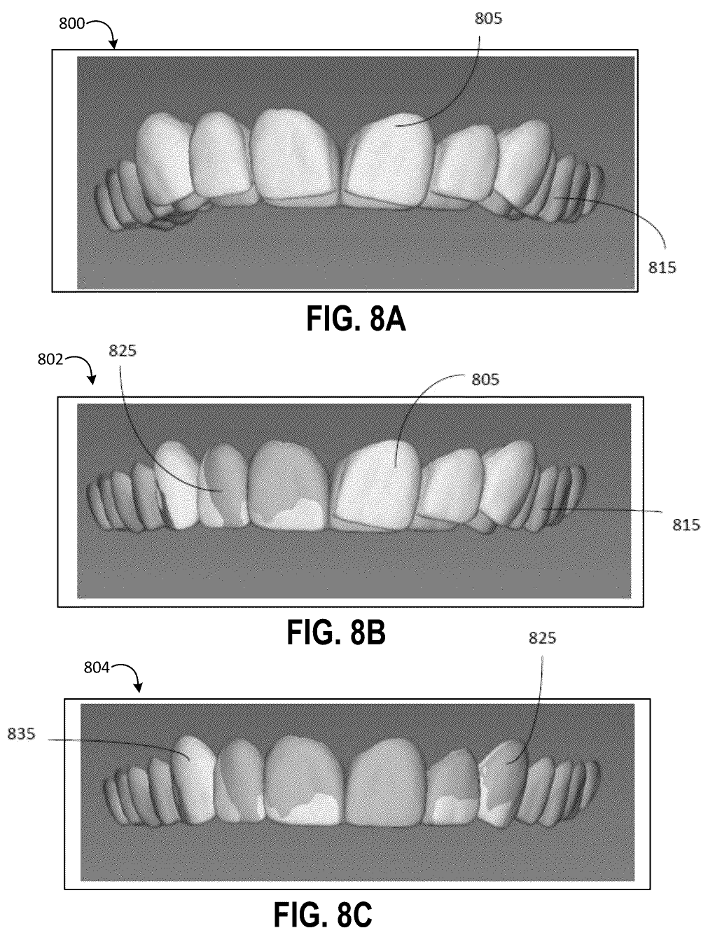

FIGS. 8A, 8B, and 8C illustrate example user interface screens (GUIs) 800, 802, and 804, respectively, that may be generated during a denture design process of automatically aligning and leveling each selected tooth library teeth. Although the GUIs 800, 802, and 804 are described in reference to designing dentures, similar GUIs can also be generated and presented to a relevant user at a user device for designing other types of dental appliances described throughout this disclosure.

The GUIs 800, 802, and 804 illustrate leveling and alignment of selected tooth libraries on a digital denture model for a particular patient. For example, the GUI 800 in FIG. 8A shows a selected tooth library 805 in an initial position with regard to a digital denture model 815. The initial position can be a position at which the selected tooth library 805 is applied to the digital denture model 815 without adjusting the positioning to be a better fit. Each tooth from the selected tooth library 805 can then be automatically fitted to a corresponding tooth of the digital denture model 815 by automatically aligning the tooth with an arch of the digital denture model 815 and leveling the tooth with respect to an occlusal plane.

The GUI 802 in FIG. 8B shows partial completion of the process for aligning the selected tooth library 805 with the digital denture model 815, where the teeth on a left side of the digital denture model 815 have been leveled and aligned to a best-fit position 825, and the teeth of the selected tooth library 805 on the right side of the digital denture model 815 are not yet fitted.

The GUI 804 in FIG. 8C shows the completed process, where all teeth are now in the best-fit position 825, and areas that the teeth of the selected tooth library 805 extend beyond boundaries of the teeth of the digital denture model 815 can be colored according to a heat map for presentation to the relevant user (for example, position 835).

As shown in the GUIs 800, 802, and 804 in FIGS. 8A, 8B, and 8C, respectively, the teeth of the digital denture model 815 can be shown in a first indicia (e.g., color, pattern, shading) and the teeth of the selected tooth library 805 can be shown in a second, different indicia. The first indicia can be a color, such as orange or green. The second indicia can be a color, such as white. Various other indicia can be used to visually represent to the relevant user how well aligned and/or positioned the selected tooth library 805 teeth are relative the teeth of the digital denture model 815.

FIG. 9A illustrates example libraries of digital denture teeth that can be automatically selected by a computer system and used to design a digital denture model for a patient. More particularly, FIG. 9A shows a first set of teeth from a tooth library 1303, a second set of teeth from a tooth library 1307, and a third set of teeth from a tooth library 1309. Although some dimensional differences may be detected upon visual inspection of the three sets of teeth 1303, 1307, 1309, full dimensional differences between the sets of teeth are visualized in FIG. 9B.

FIG. 9B illustrates differences between the digital denture teeth from the tooth libraries 1303, 1307, and 1309 of FIG. 9A with an overlay color map 1311. The overlay color map 1311 can be used to show differences between each set of teeth from the selected tooth libraries. For example, the first set of teeth from the tooth library 1303 can be presented in a first indicia (e.g., a red color), the second set of teeth from the tooth library 1307 can be presented in a second indicia (e.g., a green color), and the third set of teeth from the tooth library 1309 can be presented in a third indicia (e.g., a purple color). Differences in shape, position, orientation, and/or size of the first, second, and third sets of teeth can have various impacts on patient acceptance of the dentures or other dental appliance, such as an outsized impact on patient acceptance of the dentures, as the sets of teeth can impact look, feel, and function of the resulting dentures or other dental appliance.

FIG. 10 is a system diagram of one or more components that can be used to perform the techniques described herein. The computer system 102, user device(s) 108, static tooth library data store 106, a patient information data store 1200, and scanning devices 1230A-N can communicate (e.g., wired, wirelessly), via the network(s) 110.

The computer system 102 can include processor(s) 1202, memory 1204, the tooth library selection engine 104, an auto-design denture setup engine 1208, a perfect smile setup engine 1209, an auto-design implant setup engine 1211, an optional auto-design engine 1206, an output generator 1210, a model training engine 1212, and a communication interface 1214. The processor(s) 1202 can be configured to receive and execute instructions to perform one or more of the operations described herein. The memory 1204 can be any type of local memory/RAM configured to store instructions, data, or other information for fast and efficient retrieval and use during runtime. For example, the local memory 1204 of the computer system can store one or more models used for identifying and labelling markers in patient tooth data. The communication interface 1214 can be configured to provide communication between and amongst components of the computer system 102 and the other system components described in reference to FIG. 10.

The tooth library selection engine 104 can be configured to identify markers in tooth data, determine tooth and/or cavity measurements based on the markers, select a subset of tooth libraries based on the measurements, score each tooth library in the subset using a fitting algorithm, and/or select a candidate tooth library for use in designing a dental appliance for a particular patient. The engine 104 can receive patient tooth data from the user device(s) 108 and/or the scanning device(s) 1230A-N. The scanning devices 1230A-N can include but are not limited to intraoral scanners, computed tomography (“CT”) scanning devices, cone beam computed tomography (“CBCT”) scanning devices, and/or other types of 3D imaging devices that may be used to capture images and other scan data of patients' mouths, dentition, jaws, and/or faces.

In some implementations, the patient tooth data can be previously captured using the scanning device(s) 1230A-N, then stored in the patient information data store 1200 as patient tooth data 1218A-N. The data store 1200 can be the same as or similar to the static tooth library data store 106. The patient tooth data 1218A-N can include, but is not limited to, patient information (e.g., age, gender, teeth condition, dental appliance type, procedure, and/or history, and/or demographic information), 3D image data (e.g., captured by the scanning devices 1230A-N), 2D image data (e.g., converted from the 3D image data by the computer system 102, such as the tooth library selection engine 104), teeth scans (e.g., as generated or captured by the scanning device(s) 1230A-N or other teeth imaging devices), teeth measurements (e.g., as provided by a relevant user and/or automatically determined by the computer system 102, such as the tooth library selection engine 104), denture design(s) (e.g., as determined by the auto-design denture setup engine 1208), veneer design(s) (e.g., as determined by the perfect smile setup engine 1211), and/or dental implant design(s) (e.g., as determined by the auto-design implant setup engine 1211).

As described herein, the engine 104 can retrieve one or more models 1232A-N from the data store 1200 for use in identifying markers in the patient tooth data 1218A-N, determining tooth measurements, and/or selecting tooth libraries for the particular type of dental appliances to design for the patient. The engine 104 can retrieve one or more of the following tooth libraries from the static tooth library data store 106 when selecting tooth libraries for the particular patient: individual-tooth libraries 1220A-N, tooth-by-type libraries 1222A-N, anterior-tooth libraries 1224A-N, posterior-tooth libraries 1228A-N, and/or grouped-tooth libraries 1226A-N. The individual-tooth libraries 1220A-N can be predefined tooth libraries for individual teeth. In other words, each tooth can have one or more corresponding tooth libraries. The tooth-by-type libraries 1222A-N can be predefined tooth libraries for each type of tooth (e.g., molars, canines, incisors, etc.). The anterior-tooth libraries 1224A-N can be predefined tooth libraries for various sets or groups of anterior teeth. The posterior-tooth libraries 1228A-N can be predefined tooth libraries for various sets or groups of posterior teeth. The grouped-tooth libraries 1226A-N can be predefined tooth libraries for various sets or groups of teeth that are adjacent to each other. Once the tooth library selection engine 104 selects a subset of tooth libraries and/or a candidate tooth library, the engine 104 can transmit the selected tooth library (or libraries) to one or more of the setup engines 1208, 1209, 1211, and/or 1208. In some implementations, as described in reference to FIG. 1A, the engine 104 can transmit the selected tooth library to the setup engine that requested tooth libraries.

For example, if dentures are being designed for the particular patient, then the auto-design denture setup engine 1208 can transmit a request for a candidate tooth library to the tooth library selection engine 104. The engine 104 can then select the candidate tooth library from the data store 106 based on identifying markers in the particular patient's tooth data, determining tooth measurements based on the markers, and selecting tooth libraries using one or more tooth selection criteria 1234A-N. The tooth selection criteria 1234A-N can include one or more criteria specific to each type of dental appliance to be designed. For example, the tooth selection criteria for designing dentures can include larger threshold ranges for tooth size measurements than the tooth selection criteria for designing veneers or dental implants/inserts.

Refer to at least FIGS. 1A and 2-4 for further discussion about operations performed by the tooth library selection engine 104.

The auto-design denture setup engine 1208 can be configured to request tooth libraries from the tooth library selection engine 104 and design dentures for a particular patient using the requested tooth libraries. The engine 1208 can implement one or more machine learning models 1232A-N, AI algorithms, and other techniques to automatically design the dentures for the patient, as described further in reference to the '217 application, which is incorporated in its entirety. Denture designs generated by the engine 1208 can include 3D digital models of the dentures for the patient, which can be stored as part of the patient tooth data 1218A-N as the denture design(s) in the patient information data store 1200. The 3D digital model(s) can then be used as part of iteratively training and/or improving the models 1232A-N used and described herein. The 3D digital model(s) can also be used to design other dentures for other patients. The 3D digital model(s) can be outputted in various GUIs generated by the output generator 1210 and presented at the user device(s) 108 of the relevant users, such as dentists and technicians. The user device(s) 108 can also receive user input indicating one or more adjustments to the 3D digital model, which can then be transmitted to the auto-design denture setup engine 1208 to modify the design of the patient's dentures.

The perfect smile setup engine 1209 can be configured to request tooth libraries from the tooth library selection engine 104 and design veneers for a particular patient using the requested tooth libraries. The engine 1209 can implement one or more machine learning models 1232A-N, AI algorithms, and other techniques to automatically design the veneers for the patient. Veneer designs generated by the engine 1209 can include 3D digital models of the veneers for the patient, which can be stored as part of the patient tooth data 1218A -N as the veneer design(s) in the patient information data store 1200. The 3D digital model(s) can then be used as part of iteratively training and/or improving the models 1232A-N used and described herein. The 3D digital model(s) can also be used to design other veneers for other patients. The 3D digital model(s) can be outputted in various GUIs generated by the output generator 1210 and presented at the user device(s) 108 of the relevant users. The user device(s) 108 can also receive user input indicating one or more adjustments to the 3D digital model, which can then be transmitted to the perfect smile setup engine 1209 to modify the design of the patient's veneers in order to enhance a smile and overall appearance of the patient.

The auto-design implant setup engine 1211 can be configured to request tooth libraries from the tooth library selection engine 104 and design dental implants, inserts, or other types of dental appliances (e.g., crowns, caps) for a particular patient using the requested tooth libraries. The engine 1211 can implement one or more machine learning models 1232A-N, AI algorithms, and other techniques to automatically design the dental implants/inserts for the patient. Dental implants/inserts designs generated by the engine 1211 can include 3D digital models of the dental implants/inserts for the patient, which can be stored as part of the patient tooth data 1218A-N as the dental implant design(s) in the patient information data store 1200. The 3D digital model(s) can then be used as part of iteratively training and/or improving the models 1232A-N used and described herein. The 3D digital model(s) can also be used to design other dental implants/inserts for other patients. The 3D digital model(s) can be outputted in various GUIs generated by the output generator 1210 and presented at the user device(s) 108 of the relevant users. The user device(s) 108 can also receive user input indicating one or more adjustments to the 3D digital model, which can then be transmitted to the auto-design implant setup engine 1211 to modify the design of the patient's dental implants/inserts.

Optionally, the computer system 102 can include the auto-design engine 1206, which can be configured to automatically design any type of dental appliance for patients using selected tooth libraries, including but not limited to dentures, veneers, and/or dental implants/inserts. The engine 1206 can perform any of the operations described in reference to the auto-design denture setup engine 1208, the perfect smile setup engine 1209, and/or the auto-design implant setup engine 1211.

The output generator 1210 can be configured to generate any of the GUIs described herein for presenting information to the user at the user device(s) 108. The GUIs can provide user-selectable options for viewing and modifying/adjusting 3D digital models of different types of dental appliances for patients. The GUIs can also output information such as selected tooth libraries for different dental appliance designs. The user can interact with the GUIs presented at their user device(s) 108 to design dental appliances for patients. Refer to the '217 application for further discussion about the GUIs that can be generated and presented to the relevant users.

The model training engine 1212 can be configured to train and/or iteratively improve one or more of the models 1232A-N using training data 1216A-N. In some implementations, the engine 1212 may also train and/or iteratively train the models 1232A-N using the patient tooth data 1218A-N and/or any of the user inputs received from the user device(s) 108, and/or any decisions made by the components of the computer system 102. Refer to FIG. 5 for further discussion about training the models 1232A-N.

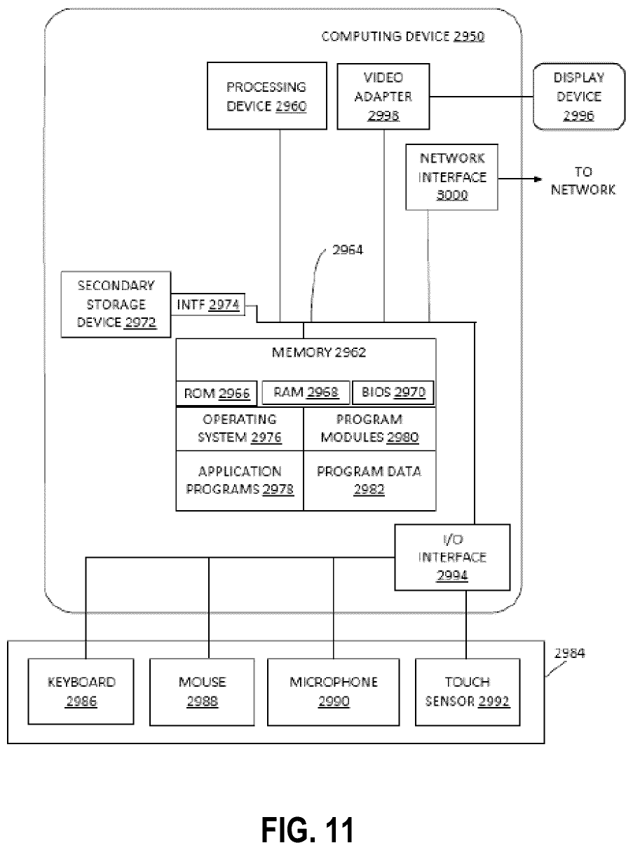

FIG. 11 is an example architecture of a computing device 2950, which can be used to implement aspects according to the present disclosure, including any of the computing systems (e.g., the computer system 102) and/or computing devices described herein, such as any type of computing device of a dental appliance design system. The computing device illustrated in FIG. 11 can be used to execute an operating system, application programs, and software modules to perform the design processes described herein.

The computing device 2950 includes, in some embodiments, at least one processing device 2960, such as a central processing unit (CPU). Examples of computing devices suitable for the computing device 2950 include a desktop computer, a laptop computer, a tablet computer, a mobile computing device (such as a smartphone, an iPod® or iPad® mobile digital device, or other mobile devices), or other devices configured to process digital instructions. The system memory 2962 includes read only memory 2966 and random-access memory 2968. A basic input/output system 2970 containing the basic routines that act to transfer information within computing device 2950, such as during start up, is typically stored in the read only memory 2966.

The computing device 2950 also includes a secondary storage device 2972 in some embodiments, such as a hard disk drive, for storing digital data. The secondary storage device 2972 is connected to the system bus 2964 by a secondary storage interface 2974. The secondary storage devices 2972 and their associated computer readable media provide nonvolatile storage of computer readable instructions (including application programs and program modules), data structures, and other data for the computing device 2950. A number of program modules can be stored in secondary storage device 2972 or system memory 2962, including an operating system 2976, one or more application programs 2978, other program modules 2980 and program data 2982.

In some embodiments, a user provides inputs to the computing device 2950 through one or more input devices 2984. Examples of input devices 2984 include a keyboard 2986, mouse 2988, microphone 2990, and touch sensor 2992 (such as a touchpad or touch sensitive display). Other embodiments include other input devices 2984. The input devices are often connected to the processing device 2960 through an input/output interface 2994 that is coupled to the system bus 2964. These input devices 2984 can be connected by any number of input/output interfaces, such as a parallel port, serial port, game port, or a universal serial bus. Wireless communication between input devices and the interface 2994 is possible as well, and includes infrared, BLUETOOTH® wireless technology, 802.11a/b/g/n, cellular, ultra-wideband (UWB), ZigBee, or other radio frequency communication systems in some possible embodiments.

In this example embodiment, a display device 2996, such as a monitor, liquid crystal display device, projector, or touch sensitive display device, is also connected to the system bus 2964 via an interface, such as a video adapter 2998. In addition to the display device 2996, the computing device 2950 can include various other peripheral devices (not shown), such as speakers or a printer.

When used in a local area networking environment or a wide area networking environment (such as the Internet), the computing device 2950 is typically connected to the network through a network interface 3000, such as an Ethernet interface or WiFi interface. Other possible embodiments use other communication devices. For example, some embodiments of the computing device 2950 include a modem for communicating across the network.

The computing device 2950 typically includes at least some form of computer readable media. Computer readable media includes any available media that can be accessed by the computing device 2950. By way of example, computer readable media include computer readable storage media and computer readable communication media. Computer readable storage media includes volatile and nonvolatile, removable and non-removable media implemented in any device configured to store information such as computer readable instructions, data structures, program modules or other data. Computer readable storage media includes, but is not limited to, random access memory, read only memory, electrically erasable programmable read only memory, flash memory or other memory technology, compact disc read only memory, digital versatile disks or other optical storage, magnetic cassettes, magnetic tape, magnetic disk storage or other magnetic storage devices, or any other medium that can be used to store the desired information and that can be accessed by the computing device 2950. Computer readable communication media typically embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are also included within the scope of computer readable media.

The computing device illustrated in FIG. 11 is also an example of programmable electronics, which may include one or more such computing devices, and when multiple computing devices are included, such computing devices can be coupled together with a suitable data communication network so as to collectively perform the various functions, methods, or operations disclosed herein.

Although a few implementations have been described in detail above, other modifications are possible. Moreover, other mechanisms for performing the systems and methods described in this document may be used. In addition, the logic flows depicted in the figures do not require the particular order shown, or sequential order, to achieve desirable results. Other steps may be provided, or steps may be eliminated, from the described flows, and other components may be added to, or removed from, the described systems. Accordingly, other implementations are within the scope of the following claims.

What is claimed is:

1. A method for selecting a candidate tooth library for auto-designing a dental appliance for a patient, the method comprising:
receiving, by a computer system, patient tooth data, wherein the patient tooth data comprises a scan of a patient's teeth;
identifying, by the computer system, one or more markers on the teeth in the patient tooth data, wherein the markers include at least opposing edges on a tooth;
determining, by the computer system, teeth measurements based on the identified markers, wherein determining the teeth measurements comprises determining a width between the opposing edges on the tooth;
selecting, by the computer system and from a static tooth library data store, a subset of tooth libraries based on the teeth measurements, wherein selecting the subset of tooth libraries based on the teeth measurements comprises:
identifying at least one tooth library having respective tooth width measurements within a threshold range of the determined width of the tooth in the patient tooth data, and
adding the identified at least one tooth library to the subset of tooth libraries;
selecting, by the computer system, a candidate tooth library from the subset of the tooth libraries based on the candidate tooth library satisfying one or more dental-appliance-fit criteria; and
returning, by the computer system, the candidate tooth library for the patient.

2. The method of claim 1, wherein identifying, by the computer system, one or more markers on the teeth in the patient tooth data comprises:
applying at least one model to the patient tooth data, the model having been trained with machine learning techniques to identify and label, as the markers, at least one of opposing tooth edges, a cusp, a cusp tip, and a marginal ridge.

3. The method of claim 1, wherein determining, by the computer system, teeth measurements based on the identified markers comprises determining lengths of incisors for at least one of the teeth.

4. The method of claim 1, wherein determining, by the computer system, teeth measurements based on the identified markers comprises determining a shape of at least one of the teeth.

5. The method of claim 1, wherein determining, by the computer system, teeth measurements based on the identified markers comprises determining at least one interproximal (IP) contact between the teeth.

6. The method of claim 1, wherein selecting, by the computer system and from a static tooth library data store, a subset of tooth libraries is based on the teeth measurements, wherein selecting the subset of tooth libraries based on the teeth measurements comprises:
aggregating tooth width measurements for the teeth in the patient tooth data;
identifying at least one tooth library having a respective aggregate tooth width measurement within a threshold range of the aggregated tooth width measurements for the teeth in the patient tooth data; and
adding the identified at least one tooth library to the subset of tooth libraries.

7. The method of claim 1, further comprising scoring, by the computer system, each tooth library in the subset of tooth libraries, wherein the computer system is configured to assign a score above a threshold value to the tooth library when the respective tooth width measurements are within a threshold scoring range of the determined width of the tooth in the patient tooth data.

8. The method of claim 7, wherein scoring further comprises assigning a score below the threshold value to the tooth library when the respective tooth width measurements exceed the threshold scoring range of the determined width of the tooth in the patient tooth data.

9. The method of claim 7, wherein the threshold scoring range is based on a type of the dental appliance being designed for the patient, the type of the dental appliance being at least one of dentures, veneers, dental implants, and dental inserts.

10. The method of claim 9, wherein a threshold scoring range for the dentures is greater than threshold scoring ranges for the veneers, the dental implants, and the dental inserts.

11. The method of claim 7, wherein selecting, by the computer system, a candidate tooth library from the subset of the tooth libraries is based on a score of the candidate tooth library satisfying the one or more dental-appliance-fit criteria.

12. The method of claim 1, wherein selecting, by the computer system, a candidate tooth library from the subset of the tooth libraries comprises:
selecting the candidate tooth library having respective incisor lengths within a threshold length range of determined incisor lengths of the teeth in the patient tooth data.

13. The method of claim 1, wherein selecting, by the computer system, a candidate tooth library from the subset of the tooth libraries comprises:
selecting the candidate tooth library having respective tooth shapes within a threshold shape range of determined shapes of the teeth in the patient tooth data.

14. The method of claim 7, wherein selecting, by the computer system, a candidate tooth library from the subset of the tooth libraries comprises:
selecting the candidate tooth library having a highest respective score amongst the subset of tooth libraries.

15. The method of claim 7, wherein selecting, by the computer system, a candidate tooth library from the subset of the tooth libraries comprises:
selecting the candidate tooth library having a respective score that exceeds a threshold score value.

16. The method of claim 1, wherein selecting, by the computer system, a candidate tooth library from the subset of the tooth libraries comprises:
removing, from the subset of the tooth libraries, at least one tooth library having respective incisor lengths that exceed a threshold length range of determined incisor lengths of the teeth in the patient tooth data;
determining whether the subset of the tooth libraries comprises more than one tooth library; and
removing, from the subset of the tooth libraries and based on determining that the subset comprises more than one tooth library, at least another tooth library having respective tooth shapes within a threshold shape range of determined shapes of the teeth in the patient tooth data.

17. The method of claim 16, wherein returning the candidate tooth library causes the computer system to auto-design a dental appliance for the patient based at least in part on the candidate tooth library, wherein the dental appliance is at least one of dentures, veneers, a dental implant, and a dental insert.

18. The method of claim 1, wherein returning, by the computer system, the candidate tooth library comprises transmitting the candidate tooth library to a user device of a relevant user for presentation in a graphical user interface (GUI) at the user device.

19. The method of claim 18, further comprising:
receiving, by the computer system from the user device, user input indicating selection of another tooth library from the subset of tooth libraries; and
auto-designing, by the computer system, a dental appliance for the patient based on the user-selected tooth library.

20. The method of claim 18, wherein presenting the candidate tooth library in the GUI comprises displaying the teeth of the candidate tooth library in a digital dental appliance model using a color map, wherein the color map illustrates differences between the teeth of the candidate tooth library and teeth of the digital dental appliance model.
